(19) United States
(12) Patent Application Publication
(10) Pub. No.: US 2025/0140358 A1
(43) Pub. Date: May 1, 2025

(54) METHODS AND SYSTEMS FOR NAVIGATING THROUGH VIRTUAL PRESENTATIONS OF A BODY PORTION GENERATED IN MEDICAL STUDIES

(71) Applicant: AI Medical AG, Zollikon (CH)

(72) Inventor: Christian Federau, Zollikon (CH)

(21) Appl. No.: 18/681,907

(22) PCT Filed: Aug. 8, 2022
(86) PCT No.: PCT/EP2022/072250

§ 371 Date: Feb. 7, 2024

(30) Foreign Application Priority Data
Aug. 9, 2021 (EP) 21190411.5

(51) Int. Cl. G16H 15/00; G16H 10/20

(52) U.S. Cl. CPC G16H 15/00; G16H 10/20

(57) ABSTRACT

A method and a system for navigating virtual presentations such as images of a body portion that are generated during medical examinations (studies). The method includes a step of providing in a user interface a list of studies, a list of presentations and a presentation of the body portion. The list of studies includes items that are characteristic for a kind of study. The list of presentations comprises items that are characteristic for a kind of presentation. The presentation displayed in the user interface depends on the item selected in the list of studies and the item selected in the list of presentations.

### BACKGROUND OF THE INVENTION

### Field of the Invention

The invention relates to the field of medical imaging technology and concerns methods and devices for selecting, ordering, displaying and/or navigating virtual presentations of a body portion. The presentations are generated during a medical examination, also called study, for example by acquiring the presentations directly or by generating them from raw data or “raw images” acquired during a study, wherein presentations of different kinds, in particular presentations optimized for visualizing different tissue or tissues and/or pathology or pathologies, are generated typically in a study. Examples of medical examinations (studies) to which the present invention relates are computer tomography (CT) examination and magnetic resonance (MR) examination. 2D images, 3D images, arrangements of 2D images or 3D images, scrollable images and movies of the body portion are examples virtual presentations of the body portion, wherein the virtual presentations are generated for and of a kind suitable for a medical purpose, in particular for assessing a medical condition.

### Description of Related Art

A medical examination (study) to which the present invention relates typically provides of a plurality of virtual presentations of a body portion, wherein the presentations are suitable to be provided to a user, for example a practitioner, for assessing a medical condition of the body portion shown in the presentations.

A virtual presentation of the body portion may include or be a 2D image of the body portion, a 3D image of the body portion, a volume rendered image of the body portion, a spatial arrangement of such images, or a movie of the body portion, for example. A 2D image generated with a top view imaging technique and a slice or projection of a 3D representation of the body portion are examples of a 2D image of the body portion. A 3D visualization of the body portion or a scrollable image of the body portion are examples of a 3D image of the body portion. A movie that shows different planes of the body portion in a consecutive manner is an example of a movie presentation of the body portion.

Amongst other things, the presentations provided in a study may differ in the clinical purpose they are optimized for. In simple terms, they may differ in the tissue or tissues and/or pathology or pathologies that may be examined best when studying a presentation.

The manner this optimization is done depends on the examination method.

For example, in the case of a CT examination, the examination includes, in many cases consists of, a scan in which raw data, usually a volume of voxels, is acquired. Different presentations of the scanned body portion are generated then by reconstructing presentations from the raw data with different reconstruction kernels or different images filters followed by windowing. During reconstruction and in particular during windowing, presentations are generated that are optimized for specific clinical purposes.

For example, in the case of a magnetic resonance examination an examination (study) typically includes, usually consists of, a plurality of acquisitions that are carried out with different settings. A setting used during an MRI acquisition is also called an MRI sequence or contrast. The acquisitions carried out using different settings result in presentations of the examined body portion that are optimized for a specific clinical purpose. In other words, the presentations of an MRI examination base on acquirements with different settings, in particular different pulse sequences and pulsed field gradients, for addressing a specific clinical purpose. Fluid Attenuated Inversion Recovery (FLAIR), T1-weighted pulse sequence, T2-weighted pulse sequence, or sequences of the diffusion-weighted imaging group are examples of different MRI sequences.

Independent of the examination method, a presentation of a study has finally a contrast that is optimized for a specific clinical purpose and that is different from the contrast of another, potential or generated, presentation of the study. Thereby, the contrast is related to the settings used during acquisition of the presentation or of data on which the presentation bases and/or to the manner the acquired data is processed.

For CT examination for example, contrasts can be defined according to the contrast-window, for example the center and width of the region of the Hounsfield scale, such as soft-tissue contrast and bone contrast.

For MR examination for example, contrasts can be defined according to the MRI sequence used during acquisition, such that FLAIR, T1-weighted, T2-weighted, proton-density and diffusion-weighted are examples of different contrasts.

In more general terms, each presentation of a study can be related to at least one characteristic used during generation of the presentation, in particular during acquisition of raw data or the presentation and/or during processing the raw data on which the presentation bases, said characteristic being representative for a specific kind of presentation, in particular for the clinical purpose the presentation is optimized for.

It is an insight of the invention, that said characteristic can be used in an efficient and resource-friendly manner for selecting, ordering, displaying and/or navigating the presentations of a plurality of studies.

Medical images software according to the state-of-the-art typically present the studies and presentations in chronological order as a list, with a name (for example the sequence name for MR, the reconstruction kernel name for CT) and/or a small representative image. This manner of presenting the studies and presentations is highly inefficient because the practitioner must first open the study she/he is interested in, then find the presentation she/he is interested in, and then use some kind of display tool, for example by selecting the presentation or by using a drag and drop function. It is evident that the practitioner, by this, spends a significant amount of time searching for the presentation she/he wants to look at, and that the practitioner has to repeat these working steps every time she/he wants to look at a new presentation or a new study, as it is in particular the case if different presentations and/or different studies need to be compared for assessing a medical condition.

### SUMMARY OF THE INVENTION

It is an object of the invention to provide methods and devices that overcome at least some of the drawback of methods and devices according to the state-of-the-art.

In particular, it is an object of the invention to provide a method and a system that supports a practitioner in analyzing the presentations of a body portion generated during a medical examination (study). In particular, it is an object of the invention to provide a method and a system for navigating and/or selecting and displaying in an efficient manner the presentations of a study or studies of a selected patient.

In particular, it is an object of the invention to provide a method and a system for navigating, in particular for switching a displayed presentation to another presentation corresponding to another study and/or another kind of presentation, in an efficient manner.

Further, it is an object of the invention to provide a method for ordering studies and their presentations in a way to further improve the efficiency of the selection, display, and navigation of the presentations by the practitioner. Therefore, a method for creating unique identifiers for virtual presentations of a body portion, the virtual presentations being generated in medical studies, is provided.

The methods are computer-implemented methods or computer supported methods at least. Therefore, it is a further object of the invention to provide related computer programs, related computer readable mediums, related computer-readable signals, and related data carrier signals.

At least one of the objects is achieved by the invention described in the following and/or the invention claimed in the claims.

According to a first aspect of the invention, a method for at least one of selecting, ordering, displaying and navigating virtual presentations of a body portion, in particular for selecting and displaying, is provided, wherein the virtual presentations are generated during medical studies, for example during a radiologic, magnetic resonance imaging (MRI), computer tomographic (CT), sonographic, or photographic study.

The method for at least one of selecting, ordering, displaying and navigating virtual presentations of a body portion generated in medical studies, in particular for selecting a displaying, includes the following steps:
A step of providing a list of studies including at least one item that is characteristic for a kind of study.
For example, a list of studies may be generated, wherein the list of studies includes items including a description defining a corresponding study.
The size of the list of studies can be different than the number of studies available for one patient.
Usually, the items of the list of studies are unique items, this means that the list of studies includes unique items, only. In other words, each item of the list of studies appears only once in the list of studies.
Further, the list of studies is provided in a manner that each item may correspond maximally to one study related to the medical case to be examined.
In an embodiment, the items of the list of studies are suitable for ordering the studies of a patient, or a selection thereof, in chronological order.
A first kind of study may differ from a second kind of study in the date the study was generated, for example.
In an embodiment, each item of the list of studies is or includes the date of a corresponding study, or the items represent an increasing or decreasing numbering.
The list of studies can be provided in an automated manner or it can be defined by a user, for example a practitioner.
In an embodiment, the list of studies is provided in an automated manner. For example, upon selecting a patient by the user, the list of studies is generated by reading the study dates of the studies available for the user and optionally for the body portion to be examined.
A step of providing a list of presentations including at least one item that is characteristic for a kind of presentation.
For example, a list of presentations may be generated, wherein the list of presentations includes items including a description defining a corresponding presentation.
A first kind of presentation may differ from a second kind of presentation in the settings used during acquisition of the presentations or raw data and/or features used during processing of the raw data, for example as disclosed above with respect to CT and MR examination.
The size of the list of presentations can be different than the number of different kinds of presentations available for the patient.
Usually, the items of the list of presentations are unique items. In other words, each item of the list of presentations appears only once in the list of presentations.
However, there may be embodiments of the method in which more than one list of presentations is provided, for example one list of presentations for each study assigned to an item of the list of studies. In this case, an item in a first list of presentation is usually identical to an item in a second list of presentations, at least if the kind of presentation to which the item refers has been generated in both studies. In other words, the list(s) of presentations provided during the method may include items that are unique within a specific list of presentations, but the items may not be unique when considering another list of presentations that is provided during the method.
In an embodiment, the items of the list of presentations refer to at least one of the settings available or used during acquisition of raw data or presentations and/or features available or used during processing the acquired raw data or the acquired presentations. Then, a presentation that is generated by using a specific setting or specific settings and/or a specific feature or specific features can be identified and accessed if the list of presentations includes an item related to the specific setting or specific settings and/or to the specific feature or specific features and the presentation was assigned to said item. In other words, the items of the list of presentations are suitable for identifying different presentations, this means different kinds of presentations.
The step of assigning presentations to an item of the list of presentations is described below in detail.
In an embodiment, the items are or include hints towards the settings and/or features used for optimizing a presentation for a specific clinical purpose.
In particular, the items, more precisely their labeling, may name or indicate the available or used contrasts in the sense as disclosed above. In the case of CT and MR examination, the label of the item may include terms such as FLAIR or T1-weighted for MRI, or soft-tissue or bone window for CT.
In an embodiment, the list of presentations is a list of contrasts.
The list of presentations, in particular if it is a list of presentations that is appropriate for a specific type of study and/or a specific medical case, can be provided in an automated manner or it can be defined by the user.
In an embodiment, the list of presentations is provided in an automated manner, for example by reading out meta data.
A step of assigning a study to an item of the at least one item of the list of studies. Usually, more than one study is assigned to the list of studies if a plurality of studies for the patient and the body portion to be examined is available. In particular, all studies of the patient and the body portion to be examined that are available may be assigned to an item of the list of studies or a selection of said studies.
The assignment of studies to the list of studies is such that maximal one study is assigned to each item of the list of studies. Typically, one study is assigned to every item of the list of studies.
A step of assigning a presentation, for example each presentation or a selection of the presentations, provided in the study or, as the case may be, studies assigned to an item of the at least one item of the list of studies to an item of the at least one item of the list of presentations.
The assignment of presentations to the lists of presentations (to the lists of presentations if more than one list of presentations is provided, for example one for each study assigned to an item in the list of studies) is such that maximal one presentation is assigned to each item of the (each) list of presentations. However, there may be one or more items to which no presentation is assigned in cases in which no assignment is possible. In the example that the items of the lists of presentations concern contrasts, one of the contrasts might for example not have been used for generating a presentation.
In summary, the presentations available for a patient can be assigned to one of the items of the list of studies or to none by assigning or not assigning the study to which a presentation belongs to an item of the list of studies, and to one of the items of a list of presentations or to none by assigning or not assigning the presentation to an item of the list of presentations.
In an embodiment, a mechanism is provided to ensure that maximal one study of the patient is assigned to an item of the list of studies and that maximal one presentation is assigned to an item of the list of presentations. An example of such a mechanism is if there is more than one presentation that could be assigned to one item of a list of presentations, then the presentation with the later date is assigned.
In an embodiment, a set of rules is used in the step of assigning a study to an item of the list of studies and/or in the step of assigning a presentation to an item of the list of presentations.
For example, a list of characteristics, this means of kinds of studies and/or of kinds of presentations, that are to be assigned (list of including characteristics in the following) can be provided, for example by the user.
The method according to this embodiment can include a step of scanning the name of a study and/or of a presentation or scanning other metadata related to the study and/or presentation for a characteristic, in particular for a characteristic comprised in the list of including characteristics.
The assignment of the study or presentation can then depend on whether the scanned name or metadata includes at least one characteristic that corresponds to at least one characteristic of the list of including characteristics.
Alternatively or in addition, a list of characteristics, this means of kinds of studies and/or of kinds of presentations, that are not to be assigned (list of excluding characteristics in the following) can be provided and used in the step of assigning a study and/or in the step of assigning a presentation.
In embodiments, the characteristics of the list of including characteristics and/or of the list of excluding characteristics as well as the characteristic or characteristics for which the name or metadata is scanned are given as terms, this means as a sequence of letters and/or numbers. In other words, the list of including characteristics may be a list of including terms and the list of excluding characteristics may be a list of excluding term.
In an embodiment, the scanned name or metadata must include any term comprised in the list of including terms or a subset thereof for the study or presentation related to the name or metadata being assigned.
In an embodiment, a plurality of lists of including terms is provided and the scanned name or metadata must comprise, for each of the provided lists of including terms, at least one term that is comprised in the list of including terms for the study or presentation related to the name or metadata being assigned.
For example, in case the items of the list of presentations relate to contrasts, the assignment can be determined by scanning the name, for example the sequence name in the case of MR examination, of the presentation, the name is normally saved in the header of the presentation, and by assigning the presentation if a term found during scanning corresponds to an item of a predefined list of including terms (contrasts), while at the same time the term found corresponds to none of the items of a list of excluding terms (contrast).
Alternatively or in addition, a model of an artificial intelligence (AI), such as a neural network, may be used in the step of assigning a study to an item of the list of studies and/or in the step of assigning a presentation to an item of the list of presentations.
In particular, the model of the AI may be trained to classify a presentation according to the items of the list of presentations. For example, the model may be trained to recognize the contrast of the presentation.
In an embodiment, the method includes a step of correcting the assignments done according to any embodiment disclosed by the user. Therefore, a user interface, in particular the user interface according to any embodiment disclosed below, may include tools configured for correcting assignments by the user, for example a pop-up view, such as a preference panel.
A step of providing to a user via a user interface, the list of studies or an adapted list of studies and the list of presentations or an adapted list of presentations.
The adapted list of studies may include only the items of the list of studies to which a study was assigned and/or it may include only items related to studies selected by the user. In addition or alternatively, the adapted list of studies may be organized (ordered) differently than the list of studies.
The adapted list of presentations may includes only the items of the list of presentations to which a presentation was assigned and/or it may include only items related to kind of presentations selected by the user. In addition or alternatively, the adapted list of presentations may be organized (ordered) differently than the list of presentations.
In embodiments in which more than one list of presentations is provided during the method, the lists of presentations may include the same set of items, independent whether a presentation was assigned to an item or not. This makes navigating through different studies and identification of the presentation displayed in the image viewer more intuitive.
In embodiments in which more than one list of presentations is provided, for example one for each study assigned to an item of the list of studies, one, in particular only one, of the provided lists of presentations may be provided to the user via the user interface.
For example, the list of presentations that is provided for the most recent study assigned to an item of the list of studies is provided to the user in an automated manner. The item of the list of study corresponding to the most recent study may be highlighted for indicating that the list of presentations corresponding to the most recent study is provided to the user, when the list of studies is provided to the user.
The user interface includes an image viewer, a first view for the list of studies or the adapted list of studies, and a second view for the list of presentations or the adapted list of presentations.
The first view and the second view are configured to allow the selection of an item of the list shown in the first view and the list shown in the second view, respectively.
The first view, the second view and the image viewer are configured for the presentation to be displayed in the image viewer that belongs to the study and kind of presentation indicated by the item selected in the list displayed in the first view and the item selected in the list displayed in the second view.
A step of selecting a presentation to be displayed in the image viewer by selecting an item of the list shown in the first view and by selecting an item of the list shown in the second view.
The selected presentation to be displayed is displayed immediately after selection and in an automated manner, usually.
Usually, the items of the list shown in the second view are linked to the assigned presentations of the study that is assigned to the selected item of the list shown in the first view.
This also means that the presentation to which an item of the list shown in the second view refers depends on the item selected in the list shown in the first view. This is in particular the case if the item of the list shown in the second view refers to a kind of presentation that is present in a first study assigned to a first item of the list shown in the first view and in a second study assigned to a second item of the list shown in the in the first view.
Further, this means that the presentation to which an item of the list shown in the second view refers changes if the item selected in the list shown in the first view changes.
In embodiments in which a list of presentations is provided for each study assigned to an item of the at least one item of the list of studies and in which the presentations of an assigned study or a selection of the presentations of the assigned study are assigned to the list of presentations provided for said study, the (adapted, as the case may be) list of presentations that is provided to the user is the (adapted) list of presentations provided for the study selected in the (adapted, as the case may be) list of studies shown in the first view.
In other words, the list shown in the second view is the list of presentations that corresponds to the study assigned to the selected item of the list shown in the first view.
Due to the fact that the presentation displayed in the image viewer belongs to the study and kind of presentation indicated by the item selected in the list displayed in the first view and the item selected in the list displayed in the second view, a change in the item selected in the list displayed in the first view, this means in the (adapted, as the case may be) list of studies, causes a change of the presentation displayed in the image viewer to a presentation belonging to the newly selected study and being of the same kind, this means having the same characteristic with respect to the characteristics indicated by the items of the list shown in the second view, this means the (adapted, as the case may be) list of presentations, (no change of the item selected in the list shown in the second view) as the presentation displayed before changing the item selected in the list shown in the first view.
Due to the fact that the presentation displayed in the image viewer belongs to the study and kind of presentation indicated by the item selected in the list displayed in the first view and the item selected in the list displayed in the second view, a change in the item selected in the list displayed in the second view, this means the (adapted, as the case may be) list of presentations, causes a change of the presentation displayed in the image viewer to a presentation of the newly selected kind of presentation and belonging to the same study (no change of the item selected in the list shown in the first view) as the presentation displayed before changing the item selected in the list shown in the second view.
In an embodiment, the selection of another item of the list shown in the first view than the one currently selected occurs at a fixed item in the list shown in the second view, and the selection of another item of the list shown in the second view than the one currently selected occurs at a fixed item in the list shown in the first view.
For example, in the case that the items of the list of presentations correspond to contrasts, the navigation in the list of studies results in displaying presentations having the same contrast but belonging to different studies. This results in an efficient navigation for the user, permitting the comparison of presentations with the same contrast of different studies with a one-click operation, for example using a keyboard shortcut, pressing on a toolbar item, or selecting an item in the first view.
For example, all the available items of the list of studies might be displayed as a selectable list in the first view. For example, the selectable list may display all available exam dates of a currently selected patient. The available exam dates may be displayed in chronological order. The item currently selected in the list shown in the first view may be highlighted. Similarly, all the available items of the list of presentations might be displayed as a selectable list in the second view. For example, the selectable list may display all available contrasts of the currently selected study.
The item currently selected in the list displayed in the second view may be highlighted. The user may then change the currently selected item in both the list provided in the first view and the list provided in the second view by clicking on another item of the lists. The presentation corresponding to the selected item of the list shown in the first view and to the selected item of the list shown in the second view is displayed in the image viewer, and the user might display another presentation corresponding to another item of the list shown in the first view and/or of the list shown in the second view by clicking on that specific item in the corresponding list.
Alternatively, or in addition, to selecting an item of the list shown in the first view and/or of the list shown in the second view by clicking on the item, the user interface may include a toolbar item configured to navigate in the list shown in the first view and to change the currently displayed study. In particular, the toolbar item may be configured to move up and/or down in the list shown in the first view.
Similarly, the user interface may include a toolbar item configured to navigate in the list shown in the second view and to change the currently displayed kind of presentation. In particular, the toolbar item may be configured to move up and/or down in the list shown in the second view.
Alternatively, or in addition, a system on which the method is carried out may be configured to accept one or more keyboard shortcuts that permit to navigate in the list shown in the first view and/or the list shown in the second view and to change the currently selected item of the list shown in the first view and/or of the list shown in the second view. In particular, the system may be configured to accept one or more keyboard shortcuts for moving up and/or down in the list shown in the first view and/or the list shown in the second view.

In an embodiment, the step of assigning studies to items of the list of studies, the step of assigning presentations to items of the lists of presentations, and the step of providing to a user the (adapted, as the case may be) list of study and the (adapted, as the case may be) list of presentations are automated steps. This means these steps are carried out in an automated manner, except an optional initialization by the user.

Optionally, the step of providing a list of studies and/or the step of providing a list of presentations may be an automated step.

Optionally, at least one of the step of providing a list of studies and the step of providing a list of presentations may be a semi-automated step. For example, at least one of a proposal for a list of studies and a proposal for a list of presentations may be provided to the user in an automated manner and the user may adapt or approve the proposed list of studies and/or the proposed list of presentations.

In an embodiment, the list shown in the first view, this means the (adapted, as the case may be) list of studies provided to the user, is an ordered (organized) list, and the list shown in the second view, this means the (adapted, as the case may be) list of presentations provided to the user, is an ordered (organized) list.

A list is ordered (organized) when it is given an order, for example through an automatic ordering method or through user interactions.

For example, the list of studies may be ordered in chronological ascending or descending study date, this means the date the study was generated.

For example, the list of presentations may be assigned a fixed order. In the case that the items of the list of presentations correspond to contrasts, the fixed order may be given by placing the FLAIR first, then the T1-weighted second, etc., for example.

In an embodiment, the user interface may provide a tool for amending the order of the list of studies and/or the order of the list of presentations.

Usually, at least the presentations assigned to an item of the list of presentations are coregistered to one another or to a reference presentation. This means that the presentations have been or are transformed in a manner that the similarity of the presentations is maximized with respect to one another or with respect to a reference presentation.

For example, the presentations are coregistered to a reference presentation, such as a template presentation, wherein the template presentation can be one of the presentations assigned (or any other presentation generated for the body portion), and hence to one another or the presentations are coregistered directly to one another. Coregistration might improve the ease to compare presentations.

Co-registration may be carried out with respect to presentation (“image”) orientation, presentation position (image position, slice number), position and orientation of the body portion shown, zoom, presentation (“image”) contrast etc.

For example, coregistration may include a step of transforming a presentation to the template presentation, and a step of resampling the transformed presentation to the template presentation, for example using interpolation, so that the voxel positions of the transformed presentation match the voxel positions of the template presentation.

Thanks to co-registration, a replacement presentation that replaces a presentation that is displayed actually in the image viewer may be nearly identical in terms of presentation orientation, presentation position (slice number), position and orientation of the body portion shown, zoom, presentation (“image”) contrast etc. In other words, the replacement presentation is nearly identical to the actually displayed presentation, except for differences that originate from the fact that the replacement presentation belongs to a different study and/or a different kind of presentation than the presentation that is displayed actually.

In an embodiment at least the presentations assigned to an item of the list of presentations are coregistered to a reference presentation, wherein the reference presentation is determined by an algorithm from a set of presentations that includes all presentations assigned to an item of the list (or lists, as the case may be) of presentations. For example the reference presentation is determined by the algorithm from all presentations of all studies concerning a specific body portion of a specific patient.

For example, the reference presentation may be determined in an automated manner by using a set of rules or by a machine learning algorithm.

Alternatively, the reference presentation may be determined by the user or a reference presentation determined in an automated manner may be corrected by the user. In this case, the user may be different from the user that examines the presentations. For example, it may be the person that is responsible for operating the medical imaging system used for generating the studies.

The reference presentation can be modified by the user, for example through a selection in a preference panel.

This reference presentation might for example be defined as a specific contrast, such as the FLAIR, of the current study.

In an embodiment, the method includes at least one of the following steps carried out by the user, in particular by the user interface including tools allowing the user to carry out at least one of the following steps:
A step of replacing the presentation displayed in the image viewer with a replacement presentation that corresponds to a presentation assigned to another item of the list shown in the second view but to the same item of the list shown in the first view.
The step of replacing may include activating the item to which the replacement presentation was assigned by clicking on the item, by pressing a toolbar item, or by using a keyboard shortcut, for example.
In particular, the replacement presentation corresponds to a presentation assigned to the item arranged in the list shown in the second view precedingly or subsequently to the item to which the presentation to which the presentation displayed in the image viewer is assigned.
In other words, the user can initiate a replacement of the presentation displayed in the image viewer with a replacement presentation that is a presentation of the same study as the presentation to be replaced (this means the presentation currently displayed in the image viewer). The replacement presentation can be assigned to the item that is arranged in the list shown in the second view one position above or one position below the item to which the presentation to be replaced is assigned. This also means that the sequence of replacements may occur in a predefined manner if the list shown in the second view looks always the same.
A step of replacing the presentation displayed in the image viewer with a replacement presentation that corresponds to a presentation of a study assigned to another item of the list shown in the first view but to the same item of the list shown in the second view.
The step of replacing may include activating the item to which the replacement presentation was assigned by clicking on the item, by pressing a toolbar item, or by using a keyboard shortcut, for example.
In particular, the replacement presentation corresponds to a presentation of the study assigned to the item arranged in the list shown in the first view precedingly or subsequently to the item to which the study to which the presentation displayed in the image viewer is assigned.
In other words, the user can initiate a replacement of the presentation displayed in the image viewer with a replacement presentation that is a presentation of the same kind as the presentation to be replaced (this means the presentation currently displayed in the image viewer). The replacement presentation can be assigned to the item that is arranged in the list shown in the first view one position above or one position below the item to which the presentation to be replaced belongs. This also means that the sequence of replacements may occurs in a predefined manner if the list shown in the first view is an ordered always in the same manner.
A step of switching back and forth between displaying in the image viewer a first presentation and a second presentation, wherein the first presentation is a presentation assigned to a first item in the list shown in the second view and the second presentation is a presentation assigned to a second item in the list shown in the second view. The first presentation and the second presentation are usually assigned to the same item of the list shown in the first view, this means they belong to the same study.
The step of switching back and forth may include activating items by clicking on the item, by pressing a toolbar item, or by using a keyboard shortcut, for example.
A step of switching back and forth between displaying in the image viewer a first presentation and a second presentation, wherein the first presentation is a presentation of the study assigned to a first item in the list shown in the first view and the second presentation is a presentation of the study assigned to a second item in the list shown in the first view. The first presentation and the second presentation are usually assigned to the same item of the list shown in the second view, this means they are of the same kind of presentations.
The step of switching back and forth may include activating items by clicking on the item, by pressing a toolbar item, or by using a keyboard shortcut, for example.

A tool, such as a toolbar item of the user interface or a keyboard shortcut, may be available in the step of switching back and forth according to the above third or forth bullet point. The tool may be configured to perform the replacement of the presentation displayed in image viewer in a toogle way, this means a replacement of the displayed presentation when using the tool and a replacement back to the initially displayed presentation when using the tool again.

Such a “toogle tool” may be used for example to switch back and forth between the current study and the immediate previous study to compare and analyse if a region of interest, such as a medical lesion, is modified in the current study compared to the previous study.

Alternatively or in addition, such a “toogle tool” may be used to switch back and forth between presentation belonging to the same study and having different contrast to assess for example a region of interest or a medical lesion in different contrasts. An important example is, while assessing a medical lesion on the FLAIR contrast, to switch to a contrast-enhanced contrast to verify if the medical lesion uptakes contrast or not, and switch back to the FLAIR contrast to continue the examination of the rest of the presentations.

According to a second aspect of the invention, a method for navigating through virtual presentations of a body portion is provided, wherein the virtual presentations are generated during medical studies, for example during a radiologic, magnetic resonance imaging (MRI), computer tomographic (CT), sonographic, or photographic study.

Again, as disclosed with respect the first aspect of the invention, the virtual presentations, in particular the virtual presentations having attributed a first or second identifier as described in detail below, are coregistered, in particular to one another, to one of the virtual presentations, or to a reference presentation.

For example, the virtual presentations having attributed a first or second identifier are coregistered to a reference presentation, wherein an algorithm determines the reference presentation from a set of virtual presentations that includes all virtual presentations having attributed a first or second identifier.

The method for navigating through virtual presentations of a body portion, wherein the virtual presentations are generated in medical studies, includes the following steps:
A step of providing a list of studies including at least one item that is characteristic for a kind of study.
The step of providing a list of studies as well as the provided list of studies may be according to any embodiment disclosed with respect to the method according to the first aspect invention.
In particular, the list of studies includes unique items.
A step of providing a list of presentations including at least one item that is characteristic for a kind of presentation.
The step of providing a list of presentations as well as the provided list of presentations may be according to any embodiment disclosed with respect to the method according to the first aspect invention.
In particular, the items of the list of presentations are suitable for identifying presentations of different kinds of presentation by the items referring to settings available or used during acquisition of raw data or presentations and/or by the items referring to features available or used during processing of acquired raw data or acquired presentations.
In particular, the list of presentations includes unique items only (and the list of studies includes unique items only).
A step of providing a dataset, wherein the dataset includes a study of a first kind of study including first virtual presentations and wherein the dataset includes a study of a second kind of study including second virtual presentations.
The first and second studies are medical studies according to any embodiment of medical studies disclosed above.
The first and second virtual presentations are virtual presentations according to any embodiment of virtual presentations disclosed above.
A step of attributing first identifiers to the first virtual presentations or to a selection thereof and second identifiers to the second virtual presentations or to a selection thereof.
The selection of first virtual presentations and the selection of the second virtual presentations include the first/second virtual presentations that are to be navigated. In particular, the selection of first virtual presentations includes not more than one presentation per kind of presentation given in the list of presentations, and not more than one presentation per kind of presentation given in the list of presentations.
The selection may be carried out as disclosed with respect to the step of assigning a presentation to an item of the at least one item of the list of presentations, embodiments of this step being disclosed in the method according to the first aspect.
A first identifier, this means each first identifier attributed to a first virtual presentation, includes a first sub-identifier of sub-identifiers of a first kind, wherein the sub-identifiers of the first kind represent the items of the list of studies in a unique manner, and a second sub-identifier of sub-identifiers of a second kind, wherein the sub-identifiers of the second kind represent the items of the list of presentations in a unique manner.
The sub-identifiers of the first kind are unique within the sub-identifiers of the first kind and such that each sub-identifier of the first kind represents exactly one item of the list of studies or no item of the list of studies. This is meant by the feature “wherein the sub-identifiers of the first kind represent the items of the list of studies in a unique manner” (also “sub-identifiers of a first kind that represent the items of the list of studies in a unique manner”).
The sub-identifiers of the second kind are unique within the sub-identifiers of the second kind and such that each sub-identifier of the second kind represents exactly one item of the list of presentations or no item of the list of presentations. This is meant by the feature “wherein the sub-identifiers of the second kind represent the items of the list of presentations in a unique manner” (also “sub-identifiers of a second kind that represent the items of the list of presentations in a unique manner”).
A sub-identifier of the second kind may represent no item of the list of presentations if the list of presentations, that may be, as disclosed with respect to the first aspect, a study-dependent list of presentations, does not include all items that are conceivable and for that unique sub-identifiers of the second kind are defined.
The first sub-identifier is equal for all first identifiers. This is because the first virtual presentations to which first identifiers are attributed belong to the same kind of study, namely the study of the first kind.
A second identifier, this means each second identifier attributed to a second virtual presentation, includes a third sub-identifier being a sub-identifier of the sub-identifiers of the first kind that represent the items of the list of studies in a unique manner and a fourth sub-identifier being a sub-identifier of the sub-identifiers of the second kind that represent the items of the list of presentations in a unique manner.
The third sub-identifier is equal for all second identifiers. This is because the second virtual presentations to which second identifiers are attributed belong to the same kind of study, namely the study of the second kind.
Due to this setting-up of the first and second identifiers, the first sub-identifier is different from the third sub-identifier.
In contrast to this, the second sub-identifier and the fourth sub-identifier may be equal in case the first virtual presentation to which the second sub-identifier is attributed and the second virtual presentation to which the fourth sub-identifier is attributed are of the same kind of presentations.
Further, each first identifier and each second identifier attributed to a virtual presentation is unique and attributed to not more than one presentation. This is also a direct consequence of the manner the first and second identifiers are set-up, of the manner the list of studies and the list of presentations are set-up, and, as the case may be, of the manner first virtual presentations are selected from the study of the first kind and second virtual presentations are selected from the study of the second kind, wherein first identifiers are attributed to the selected first virtual presentations only and wherein second identifiers are attributed to the selected second virtual presentations only.
In summary, the identifiers attributed to (a selection of, as the case may be) the first virtual presentations and to (a selection of, as the case may be) the second virtual presentations are such that a 2D grid is spanned, wherein the sub-identifiers of the first kind define a first axis of the grid and the sub-identifiers of the second kind define a second axis of the grid. This also means that the sub-identifiers of the first kind and the sub-identifiers of the second kind are on the same hierarchical level.
Due to the manner the identifiers attributed to (a selection of, as the case may be) the first virtual presentations and to (a selection of, as the case may be) the second virtual presentations are defined, it is possible to navigate with only one user input, for example “one click”, through the (selected, as the case may be) virtual presentations of a specific kind of study or through the (selected, as the case may be) presentations of the first and second studies that are of the same kind of presentation.
Further, it is possible with maximum two user inputs, for example not more than “two clicks”, to get from an arbitrary virtual presentation of the (selection of the, as the case may be) study of the first or second kind to any other presentation of the (selection of the, as the case may be) study of the first or second kind.
The user inputs may be given via the user interface, in particular via the list of studies provided in a first view thereof and the list of presentations provided in a second view thereof.
In an embodiment, the step of attributing first identifiers to the first virtual presentations or to a selection thereof and second identifiers to the second virtual presentations or to a selection thereof includes:
   A step of assigning the study of the first kind to its corresponding item in the list of studies and of assigning the study of the second kind to its corresponding item in the list of studies; and
   A step of assigning the first virtual presentations or the selection thereof to their corresponding items of the at least one item of the list of presentations and of assigning the second virtual presentations or the selection thereof to their corresponding items of the at least one item of the list of presentations.

The assignment of the study of the first kind to its corresponding item in the list of studies, this assignment being carried out during the step of assigning the study of the first and second kinds, may be carried out according to any embodiment of the step of assigning a study to an item of the at least one item of the list of studies disclosed with respect to the first aspect.

The assignment of the study of the second kind to its corresponding item in the list of studies, this assignment being carried out during the step of assigning the study of the first and second kinds, may be carried out according to any embodiment of the step of assigning a study to an item of the at least one item of the list of studies disclosed with respect to the first aspect.

The assignment of the first virtual presentations or the selection thereof to their corresponding items of the at least one item of the list of presentations, this assignment being carried out during the step of assigning the first and second virtual presentations or selections thereof, may be carried out according to any embodiment of the step of assigning a presentation provided in a study assigned to an item of the at least one item of the list of studies to an item of the at least one item of the list of presentations disclosed with respect to the first aspect.

The assignment of the second virtual presentations or the selection thereof to their corresponding items of the at least one item of the list of presentations, this assignment being carried out during the step of assigning the first and second virtual presentations or selections thereof, may be carried out according to any embodiment of the step of assigning a presentation provided in a study assigned to an item of the at least one item of the list of studies to an item of the at least one item of the list of presentations disclosed with respect to the first aspect.
A step of providing to a user via a user interface the list of studies or an adapted list of studies and the list of presentations or an adapted list of presentations.
The user interface includes an image viewer, a first view for the list of studies or the adapted list of studies, and a second view for the list of presentations or the adapted list of presentations.
This step of providing may be according to any embodiment disclosed with respect to the first aspect.
In particular, the user interface, the provided list of studies, the adapted list of studies, the provided list of presentations, and the adapted list of presentations may be according to any embodiment disclosed with respect to the first aspect.
A step of navigating through at least one of the first virtual presentations having attributed a first identifier, this means the first virtual presentations to which one of the above-disclosed unique first identifiers is attributed, and the second virtual presentations having attributed a second identifier, this means the second virtual presentations to which one of the above-disclosed unique second identifiers is attributed.
The navigation is done by selecting an item of the list shown in the first view, this means the list of studies provided via the first view, and by selecting an item of the list shown in the second view, this means the list of presentations provided via the second view.
The step of navigating includes further displaying a virtual presentation, wherein the displayed virtual presentation has the sub-identifier of the sub-identifiers of the first kind that represents the item selected in the list of studies provided in the first view and the sub-identifier of the sub-identifiers of the second kind that represents the item selected in the list of presentations provided in the second view.
Due to the fact that the step of navigating includes a step of selecting and a step of displaying, the step of navigating may include any features disclosed with respect to the step of selecting in the method according to the first aspect.

It is evident from the aforementioned disclosure that the generated unique identifiers to which the first and second identifiers belong, form a basis of the method according to the first aspect of the invention and a basis of the method according to the second aspect of the invention.

Therefore, according to a third aspect of the invention, a method for creating unique identifiers, in particular unique identifiers for navigating through virtual presentations of a body portion are generated. Again, the virtual presentations are generated in medical studies.

The method for creating unique identifiers includes:
The step of providing a list of studies including at least one item that is characteristic for a kind of study according to any embodiment disclosed above.
In particular, the items of the list of study are unique and characteristic for one kind of study only.
The step of providing a list of presentations including at least one item that is characteristic for a kind of presentation according to any embodiment disclosed above.
In particular, the items of the list of presentations are unique and characteristic for one kind of presentation only.
A step of generating first identifiers, wherein a first identifier includes a first sub-identifier of sub-identifiers of a first kind that represent the items of the list of studies in a unique manner and a second sub-identifier of sub-identifiers of a second kind that represent the items of the list of presentations in a unique manner.
In an embodiment, the sub-identifiers of the first kind are unique within the sub-identifiers of the first kind and they are, directly or indirectly, attributed to the items of the list of studies in a manner that one sub-identifier of the first kind is attributed to each item of the list of studies in a direct or indirect manner.
Further, according to this embodiment, the sub-identifiers of the second kind are unique within the sub-identifiers of the second kind and they are, directly or indirectly, attributed to the items of the list of presentations in a manner that one sub-identifier of the second kind is attributed to each item of the list of presentations in a direct or indirect manner.
In this case, the step of generating first identifiers may comprise:
   A step of assigning a study including virtual presentations and for which the unique identifiers needs to be generated to an item of the list of studies; and
   A step of assigning the virtual presentations of this study or a selection thereof to the corresponding items of the list of presentations.
   Thereby, not more than one virtual presentation of this study is assigned to the items of the list of presentations.
The first identifiers generated in this manner are unique.
This is in particular the case thanks to the structure of the list of studies and the list of presentations, in particular the unique items of the list of studies, each of them being characteristic for exactly one kind of study, and the unique items of the list of presentations, each of them being characteristic for exactly one kind of presentation, thanks to the composition of the sub-identifiers of the first and second kind and their link to the items of the list of studies and list of presentations, as well as thanks to the manner the first identifiers are generated for a study by assigning the study and its virtual presentations to items of the list of studies and to items of the list of presentations.
It goes without saying that the identifiers attributed to the items of the list of studies do not need to be sub-identifiers of the first kind. It is important only that there is a one-to-one relation between the identifiers attributed to the items of the list of studies and sub-identifiers of the first kind. This is one example of an indirect attribution mentioned above.
It goes without saying that the identifiers attributed to the items of the list of presentations do not need to be sub-identifiers of the second kind. It is important only that there is a one-to-one relation between the identifiers attributed to the items of the list of presentations and sub-identifiers of the second kind. This is one example of an indirect attribution mentioned above.

In embodiments, unique identifiers need to be generated for at least two studies, such as for the study of the first kind including first virtual presentations and the study of the second kind including second virtual presentations disclosed above.

Therefore, the method according such embodiments may include step of accessing a dataset, wherein the dataset includes a study of a first kind including first virtual presentations and wherein the dataset includes a study of a second kind including second virtual presentations.

In such embodiments, the method may include further a step of generating second identifiers, wherein a second identifier includes a third sub-identifier that is a sub-identifier of the sub-identifiers of the first kind and a fourth sub-identifier that is a sub-identifier of the sub-identifiers of the second kind.

Thereby, the first sub-identifier is equal for all first identifiers and the third sub-identifier is equal for all second identifiers, wherein this first sub-identifier is different from this third sub-identifier.

This is the case because the first virtual presentations to which first identifiers are attributed belong to the same kind of study, namely the study of the first kind, and the second virtual presentations to which second identifiers are attributed belong to the same kind of study, namely the study of the second kind, when the method according to this embodiment is used.

In an embodiment, the sub-identifiers of the first kind are still unique within the sub-identifiers of the first kind although there is a third sub-identifier that is a sub-identifier of the sub-identifiers of the first kind. Further, the sub-identifiers of the first kind are still, directly or indirectly, attributed to the item of the list of studies in a manner that one sub-identifier of the first kind is attributed to each item of the list of studies in a direct or indirect manner. In particular, the first sub-identifier is attributed in a direct or indirect manner to the item of the list of studies that represents the kind of the study of the first kind (in order words, that represents the first kind) and the third sub-identifier is attributed in a direct or indirect manner to the item of the list of studies that represents the kind of the study of the second kind (in order words, that represents the second kind).

Further, according to this embodiment, the sub-identifiers of the second kind are still unique within the sub-identifiers of the second kind although there is a fourth sub-identifier that is a sub-identifier of the sub-identifiers of the second kind. Further, the sub-identifiers of the second kind are still, directly or indirectly, attributed to the items of the list of presentations in a manner that one sub-identifier of the second kind is attributed to each item of the list of presentations in a direct or indirect manner. In particular, the second sub-identifier is attributed in a direct or indirect manner to the item of the list of presentations that represents the characteristic of the first virtual presentation for which the second sub-identifier is generated and the fourth sub-identifier is attributed in a direct or indirect manner to the item of the list of presentations that represents the characteristic of the second virtual presentation for which the fourth sub-identifier is generated.

In this embodiment, the step of creating unique identifiers may include the following steps instead of the above-disclosed steps of assigning a study including virtual presentations and for which the unique identifiers needs to be generated to an item of the list of studies and of assigning the virtual presentations of this study or a selection thereof to the corresponding items of the list of presentations:
A step of assigning the study of the first kind to its corresponding item in the list of studies and of assigning the study of the second kind to its corresponding item in the list of studies; and
A step of assigning the first virtual presentations or the selection thereof to their corresponding items of the at least one item of the list of presentations and of assigning the second virtual presentations or the selection thereof to their corresponding items of the at least one item of the list of presentations.

These two steps may be according to any embodiment of the related steps disclosed with respect to the first and second aspects of the invention.

In an embodiment, the method of creating unique identifiers includes:
The step of providing a list of studies including at least one item that is characteristic for a kind of study according to any embodiment disclosed above;
The step of providing a list of presentations including at least one item that is characteristic for a kind of presentation, according to any embodiment disclosed above;
The step of accessing a dataset, wherein the dataset includes the study of a first kind including first virtual presentations and wherein the dataset includes the study of a second kind including second virtual presentations;
The step of generating first identifiers for the study of a first kind according to any embodiment disclosed above, wherein the first sub-identifier is equal for all first identifiers;
The step of generating second identifiers for the study of a second kind according to any embodiment disclosed above, wherein the third sub-identifier is equal for all second identifiers, and wherein the first-sub-identifier is different from the third sub-identifier;
A step of attributing first identifiers to the first virtual presentations or to a selection thereof and second identifiers to the second virtual presentations or to a selection thereof, wherein the step of attributing includes the step of assigning the study of the first kind to its corresponding item of the list of studies and of assigning the study of the second kind to its corresponding item of the list of studies according to any embodiment disclosed above, and a step of assigning the first virtual presentations or the selection thereof to their corresponding items of the at least one item of the list of presentations and of assigning the second virtual presentations or the selection thereof to their corresponding items of the at least one item of the list of presentations according to any embodiment disclosed above.

The method for creating unique identifiers is suitable and configured for use in a method for navigating through virtual presentations, wherein the virtual presentations are generated in medical studies. Alternatively or in addition, the method for creating unique identifiers is suitable and configured for use in a method for at least one of selecting and displaying and or ordering such virtual presentations.

In an embodiment, the method for creating unique identifiers is used in a method for navigating through virtual presentations, in particular virtual presentations of at least two studies. In this case, the method for creating unique identifiers may comprise:
The step of providing a list of studies including at least one item that is characteristic for a kind of study according to any embodiment disclosed above;
The step of providing a list of presentations including at least one item that is characteristic for a kind of presentation, according to any embodiment disclosed above;
The step of accessing a dataset, wherein the dataset includes the study of a first kind including first virtual presentations and wherein the dataset includes the study of a second kind including second virtual presentations;
The step of generating first identifiers for the study of a first kind according to any embodiment disclosed above, wherein the first sub-identifier is equal for all first identifiers;
The step of generating second identifiers for the study of a second kind according to any embodiment disclosed above, wherein the third sub-identifier is equal for all second identifiers, and wherein the first-sub-identifier is different from the third sub-identifier.

Optionally, the method for creating unique identifiers may include further the step of attributing first identifiers to the first virtual presentations or to a selection thereof and second identifiers to the second virtual presentations or to a selection thereof, wherein the step of attributing includes the step of assigning the study of the first kind to its corresponding item of the list of studies and of assigning the study of the second kind to its corresponding item of the list of studies according to any embodiment disclosed above, and a step of assigning the first virtual presentations or the selection thereof to their corresponding items of the at least one item of the list of presentations and of assigning the second virtual presentations or the selection thereof to their corresponding items of the at least one item of the list of presentations according to any embodiment disclosed above.

The method for navigating through virtual presentations of a body portion according to this embodiment includes then:
The step of providing to a user via a user interface the list of studies or an adapted list of studies and the list of presentations or an adapted list of presentations according to any embodiment disclosed with respect to the second aspect of the invention.
In particular, the user interface is according to any embodiment disclosed and includes an image viewer, a first view for the list of studies or the adapted list of studies, and a second view for the list of presentations or the adapted list of presentations.
The step of navigating through at least one of the first virtual presentations having attributed a first identifier and the second virtual presentations having attributed a second identifier according to any embodiment disclosed with respect to the second aspect of the invention.
In particular, the step of navigating includes selecting an item of the list shown in the first view and selecting an item of the list shown in the second view according to any embodiment disclosed.
In particular, the step of navigating includes further displaying a virtual presentation, wherein the displayed virtual presentation has the sub-identifier of the sub-identifiers of the first kind that represents the item selected in the list of studies provided in the first view and the sub-identifier of the sub-identifiers of the second kind that represents the item selected in the list of presentations provided in the second view.

Independent of the aspect of the invention, the list shown in the first view may be an ordered (organized) list, and the list shown in the second view may be an ordered (organized) list in methods that include providing to a user via a user interface the list of studies or an adapted list of studies and the list of presentations or an adapted list of presentations, wherein the user interface includes an image viewer, a first view for the list of studies or the adapted list of studies, and a second view for the list of presentations or the adapted list of presentations.

Independent of the aspect of the invention, the virtual presentations having attributed a first or second identifier may be linked to the items of the list shown in the first view and the items of the list shown in the second view via the attributed identifier, in methods that include providing to a user via a user interface the list of studies or an adapted list of studies and the list of presentations or an adapted list of presentations, wherein the user interface includes an image viewer, a first view for the list of studies or the adapted list of studies, and a second view for the list of presentations or the adapted list of presentations.

Thereby, a specific virtual presentation of the virtual presentations having attributed a first or second identifier can be accessed and/or displayed, wherein the specific virtual presentation has the sub-identifier of the sub-identifiers of the first kind that represents the item selected in the list of studies provided in the first view and the sub-identifier of the sub-identifiers of the second kind that represents the item selected in the list of presentations provided in the second view.

Independent of the aspect of the invention, any method that includes providing to a user via a user interface the list of studies or an adapted list of studies and the list of presentations or an adapted list of presentations, wherein the user interface includes an image viewer, a first view for the list of studies or the adapted list of studies, and a second view for the list of presentations or the adapted list of presentations, may a step of providing a list of presentations for each study to be navigated in, wherein the list shown in the second view is the list of presentations or the adapted list of presentations that corresponds to the study selected by selecting an item of the list shown in the first view.

Details of this step are disclosed with respect to the first aspect of the invention. However, the details are true and may be present in methods according to the second and third aspect, too.

Independent of the aspect of the invention, the selection of another item of the list shown in the first view than the currently selected item may occur at a fixed item in the list shown in the second view, and the selection of another item of the list shown in the second view than the currently selected item may occur at a fixed item in the list shown in the first view, in methods that include providing to a user via a user interface the list of studies or an adapted list of studies and the list of presentations or an adapted list of presentations, wherein the user interface includes an image viewer, a first view for the list of studies or the adapted list of studies, and a second view for the list of presentations or the adapted list of presentations.

Details of this selection approach are disclosed with respect to the first aspect of the invention. However, said details are true and may be present in methods according to the second and third aspect, too.

As mentioned, the user interface used in methods according to the second or third aspect of the invention may be according to any embodiment disclosed with respect to methods according to the first aspect.

In particular, the user interface used in methods according to the second or third aspect of the invention may include any tools disclosed with respect to methods according to the first aspect.

For example, the user interface may include tools allowing the user to at least one of the replacement of the presentation displayed in the image viewer with a replacement presentation according to any embodiment disclosed with respect to methods according to the first aspect of the invention and switching back and forth between displaying in the image viewer a first presentation and a second presentation.

The invention relates further to a system for at least one of selecting, ordering, displaying and navigating virtual presentations of a body portion, wherein the virtual presentations are generated during medical studies, wherein the studies and virtual presentations may be as disclosed with respect to any embodiment of the method.

The system may be configured or may have components configured to carry out any step of a method according to any embodiment disclosed with respect to any aspect of the invention and/or to provide any opportunity to a user according to any embodiment disclosed with respect to a method according to any embodiment disclosed with respect to any aspect of the invention.

Likewise, the method in any embodiment disclosed with respect to any aspect of the invention may include any step related to any feature disclosed with respect to the system and/or any feature disclosed with respect to the system.

In a first embodiment, the system is a system for navigating through, alternatively or in addition for selecting and displaying, virtual presentations of a body portion generated in medical studies, wherein the system is configured to carry out a method according to any embodiment disclosed with respect to any aspect of the invention.

In particular, the system may be configured to carry out the method for navigating through virtual presentations of body portions generated in medical studies according to any embodiment disclosed.

Alternatively or in addition, the system may be configured to carry out the method for selecting and displaying (and optionally ordering and/or navigating) virtual presentations of body portions generated in medical studies according to any embodiment disclosed.

Therefore, the system includes a controller and a user interface. The user interface includes an image viewer, a first view for providing a list of studies or an adapted list of studies to a user, a second view for providing a list of presentations or an adapted list of presentations to a user, and tools for selecting an item of the list shown in the first view and an item of the list shown in the second view.

The user interface may be according to any embodiment of the user interface disclosed with respect to any embodiment of the method. In particular, the user interface may include any tool, toolbar items, buttons etc. disclosed with respect to embodiments of the method.

The first and second views as well as the list displayed in the first view and the list displayed in the second view may be according to any embodiment disclosed with respect to any method. In particular, the first and second views as well as the list disclosed in the first view and the list disclosed in the second view may be configured to generate any functionality, in particular any functionality with respect to selecting, switching, navigating and displaying, disclosed with respect to any embodiment of the method.

The controller may be configured to carry out at least one of the step of providing a list of studies in any embodiment disclosed, the step of providing a list of presentations in any embodiment disclosed, the step of assigning studies to an item of the list of studies in any embodiment disclosed, the step of assigning presentations to an item of a list of presentations in any embodiment disclosed, the step of providing the list of studies or an adapted list of studies to the user in any embodiment disclosed, and a step of providing the list of presentations or an adapted list of presentations to the user in any embodiment disclosed.

In particular, the controller may be configured to carry the steps of the method that are automated steps.

Alternatively or in addition, the controller may be configured to update information provided to the user via the user interface, such as the presentation displayed in the image viewer and/or the list provided in the second view or the links to which items of the list shown in the second view refers, in reaction to a user input.

In a second embodiment, the system is an evaluation system for virtual presentations of a body portion, wherein the virtual presentations are generated in medical studies.

The following applies in the system according to the second embodiment:
A list of studies including at least one item that is characteristic for a kind of study and a list of presentations including at least one item that is characteristic for a kind of presentation, wherein the list of studies and the list of presentations are stored in the memory.
The list of studies and the list of presentations may be and/or be generated according to any embodiment disclosed with respect to any method.
The communication unit is configured to access a dataset, wherein the dataset includes a study of a first kind including first virtual presentations and wherein the dataset includes a study of a second kind including second virtual presentations.
The study of a first kind and the study of a second kind may be and/or be generated according to any embodiment disclosed with respect to any method.
The first identifiers are attributed to the first virtual presentations or to a selection thereof and the second identifiers are attributed to the second virtual presentations or to a selection thereof.
The selection of first virtual presentations and the selection of second virtual presentations may be and/or be generated according to any embodiment of selecting virtual presentations of a study disclosed with respect to the any method.
The first and second identifiers may be and/or be created according to any embodiment disclosed with respect to any method.
In particular, a first identifier includes a first sub-identifier of sub-identifiers of a first kind that represent the items of the list of studies in a unique manner and a second sub-identifier of sub-identifiers of a second kind that represent the items of the list of presentations in a unique manner, wherein the first sub-identifier is equal for all first identifiers,
Further, a second identifier includes a third sub-identifier being a sub-identifier of the sub-identifiers of the first kind and a fourth sub-identifier being a sub-identifier of the sub-identifiers of the second kind, wherein the third sub-identifier is equal for all second identifiers, and wherein the first sub-identifier is different from the third sub-identifier.
The first to fourth sub-identifiers and the sub-identifiers of the first and second kind may be and/or be created according to any embodiment disclosed with respect to any method.
The user interface includes an image viewer, a first view for providing the list of studies or an adapted list of studies to a user, and a second view for providing the list of presentations or an adapted list of presentations to a user.
Further, the user interface is configured to receive a user input, wherein the user input includes selecting an item of the list shown in the first view, this means the list of studies provided via the first view, and an item of the list shown in the second view, this means the list of presentations provided via the second view.
The user interface may be according to any embodiment disclosed with respect to any method.
The provided (adapted, as the case may be) list of studies and the provided (adapted, as the case may be) list of presentations may be and/or be generated according to any embodiment disclosed with respect to any method.
The user interface may be configured to carry out the step of selecting according to any embodiment disclosed with respect to any method.
The controller is configured to control the evaluation system such that a virtual presentation is displayed in the image viewer, wherein the displayed virtual presentation is the virtual presentation that has the sub-identifier of the sub-identifiers of the first kind that represents the item selected in the list of studies provided in the first view and the sub-identifier of the sub-identifiers of the second kind that represents the item selected in the list of presentations provided in the second view.
The controller may be configured to carry out the step of navigating according to any embodiment disclosed with respect to any method.

The invention concerns further a computer program including instructions which, when the program is executed by a computer, cause the computer to carry out the method according any embodiment disclosed, in particular the method according to any embodiment disclosed with respect to the first, second or third aspect of the invention.

The invention concerns further a computer-readable medium having stored thereon instructions which, when executed by a computer, cause the computer to carry out the method according to any embodiment disclosed, in particular the method according to any embodiment disclosed with respect to the first, second or third aspect of the invention.

The invention concerns further a data carrier signal carrying instructions which, when executed by a computer, cause the computer to carry out the method according to any embodiment disclosed, in particular the method according to any embodiment disclosed with respect to the first, second or third aspect of the invention.

In particular, the invention concerns any reproducible computer-readable signal encoding the computer program that, when loaded and executed on a computer, causes the computer to carry out the method according to any embodiment disclosed, in particular the method according to any embodiment disclosed with respect to the first, second or third aspect of the invention.

The computer that is caused to carry out the method may be a computer or a computer network of the system in any embodiment disclosed.

The invention concerns further a method of manufacturing a non-transitory computer readable medium, including the step of storing, on the computer readable medium, computer-executable instructions which when executed by a processor of a computing system, cause the computing system to perform the method according to any embodiment disclosed, in particular the method according to any embodiment disclosed with respect to the first, second or third aspect of the invention.

### BRIEF DESCRIPTION OF THE DRAWINGS

Exemplarily embodiments of the invention are shown in the following figures. In the figures, the same reference symbol is used for identical or comparable elements. It shows:

FIG. 1 a flow chart of an exemplary embodiment of a method according to the first aspect of the invention;

FIG. 2 a flow chart of an exemplary embodiment of a method according to the second aspect of the invention;

FIG. 3 a visualization of an exemplary embodiment of a background process running during execution of a method according to FIG. 1 or 2;

FIG. 4 a visualization of the data structure resulting from the background process according to FIG. 3;

FIG. 5 an exemplary embodiment of a user interface;

FIG. 6 a visualization of an exemplary embodiment of a step of assigning studies to a list of studies;

FIG. 7 a visualization of an exemplary embodiment of a step of assigning presentations to a list of presentations;

FIG. 8 a visualization of an exemplary embodiment of the steps of assigning studies to a list of studies and of assigning presentations to lists of presentations; and

FIG. 9 a visualization of an exemplary embodiment of navigating in the presentations of a plurality of studies and of a plurality of kinds of presentations.

### DETAILED DESCRIPTION OF THE EMBODIMENTS

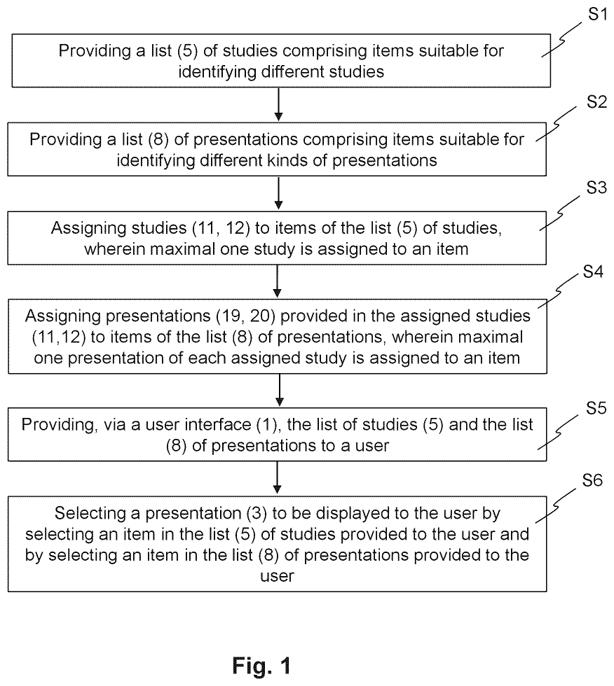

FIG. 1 shows a flow chart of an exemplary embodiment of a method according to the first aspect of the invention, wherein the method shown is a method for selecting and displaying presentations of a body portion of a patient, such as 3D representations of the body portion. The presentations are received usually from a server/archive on which the presentations after their generation in a medical examination are stored. For example, the archive is a picture archiving communication system (PACS). Alternatively, the presentations of the body portion may be received from any other server/archive, for example from the imaging system used for acquiring the presentations or raw data used to generate the presentations, or from any sever that can be in communication with the imaging system and the system used for carrying out the method.

The exemplary embodiment shown in FIG. 1 includes:
A step S1 of providing a list 5 of studies including items suitable for identifying different studies.
Items that indicate by their labeling the date a study was carried out are good examples of such items. Hence, the list of studies may be a list of dates, for example.
The list 5 of studies is usually a chronological list.
A step S2 of providing a list 8, 21, 21′ of presentations including items suitable for identifying different kinds of presentations.
Items that indicate by their labeling the contrast that is inherent to a kind of presentations due the manner the presentations of the kind of presentations are generated are good examples of such items. Hence, the list of presentations may be a list of contrasts, for example.
The list 8, 21, 21′ of presentations is usually an organized list. For example, it is organized according to a workflow used by a practitioner during a medical evaluation of the body portion.
A step S3 of assigning studies 11, 12 to items of the list 5 of studies, wherein maximal one study is assigned to an item.
For example, every study that refers to the body portion of the patient, said body portion being the body portion to be evaluated, may be assigned each to exactly one item of the list 5 of studies but never to an item of the list 5 of studies to which another study is assigned.
It goes without saying that the study that is assigned to an item of the list 5 of studies is the study that corresponds to, for example has, the feature (characteristic) indicated by item to make the item suitable for identifying different studies.
In other words, the studies 11, 12 are assigned to their corresponding items of the list 5 of studies, this means to the items of the list 5 of studies suitable for identifying these studies 11, 12.
A step S4 of assigning presentations 19, 20 to items of the list 8, 21, 21′ of presentations, wherein the presentations belong to studies 11, 12 assigned to items of the list 5 of items and wherein maximal one presentation of each assigned study is assigned to an item of the list 8, 21, 21′ of presentations.
It goes without saying that the presentations that are assigned to an item of the list 8, 21, 21′ of presentation are presentations of the kind to which the item refers. In other words, the presentations that are assigned to an item of the list 8, 21, 21′ of presentations have the feature (characteristic) that corresponds to the feature (characteristic) indicated by the item to make the item suitable for identifying different kinds of presentations.
For example, for each study assigned to an item of the list 5 of studies all presentations may be assigned to exactly one item of the list 8, 21, 21′ of presentations but never to an item of the list 8, 21, 21′ of presentations to which another presentation of the same study is assigned.
A step S5 of providing, via a user interface 1, the list 5 of studies or a list of available studies and the list 8, 21, 21′ of presentations or a list of available kinds of presentations to a user, wherein the user interface allows for the selection of one item in the provided list 5 of studies and of one item in the provided list 8, 21, 21′ of presentations, wherein the selections can be changed.
The list of available kinds of presentations or at least its layout may be study-dependent.
Further, the user interface 1 is configured to display the presentation belonging to the study selected by selecting an item in the provided list 5 of studies and being of the kind selected by selecting an item in the provided list 8, 21, 21′ of presentations.
A step S6 of selecting a presentation 3 to be displayed to the user by selecting an item in the list 5 of studies provided to the user and by selecting an item in the list 8 of presentations provided to the user.
In particular, the user selects in a consecutive manner the presentations she/he wants to see by selecting items in the provided list 5 of studies and/or in the provided list 8, 21, 21′ of presentations. In other words, the user navigates in a very simple and intuitive manner through the presentations available for the body portion of the patient to be evaluated.

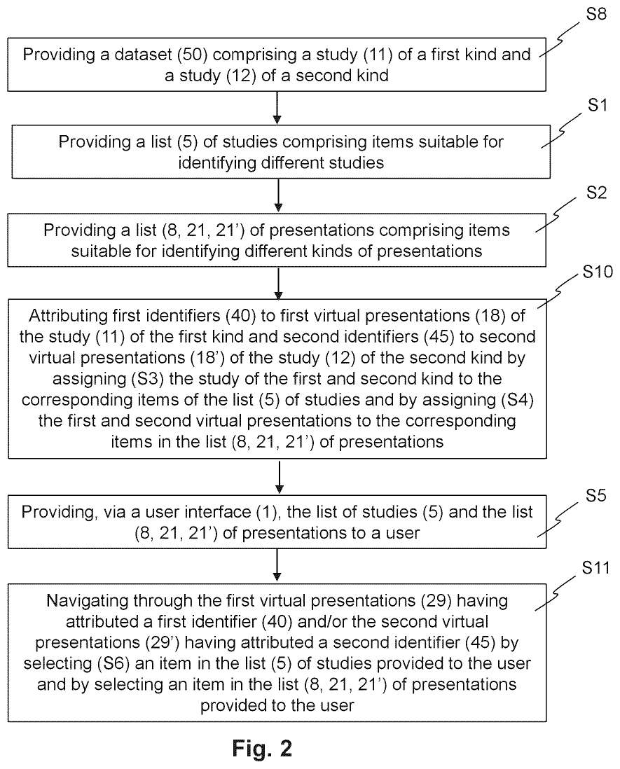

FIG. 2 shows a flow chart of an exemplary embodiment of a method according to the second aspect of the invention, this means of a method for navigating through virtual presentations of a body portion of a patient, such as 3D representations of the body portion.

Again, the presentations are received usually from a server/archive on which the presentations after their generation in a medical examination are stored and the method shown includes a step S8 of providing (for example accessing, receiving or retrieving) a dataset 50.

The dataset 50 provided includes a study 11 of a first kind and a study 12 of a second kind. The study 11 of the first kind includes the virtual presentations of the body portion acquired during this study 11. The virtual presentations of the study 11 of the first kind are called first virtual presentations 18 in this text. The study 12 of the second kind includes the virtual presentations of the body portion acquired during this study 12. The virtual presentations of the study 12 of the second kind are called second virtual presentations 18′ in this text.

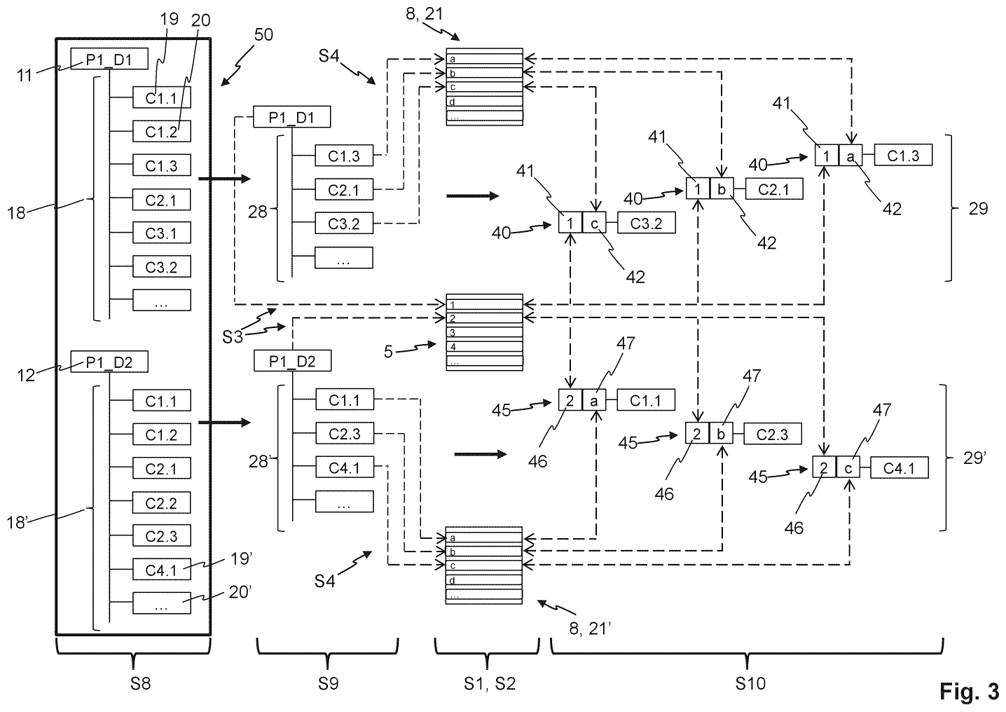

The study 11 of the first kind and its first virtual presentations 18 as well as the study 12 of the second kind and its second virtual presentation 18′ are processed then in the following manner for allowing an efficient navigation through the first and second virtual presentations, in particular efficient with respect to a medical assessment of the first and second virtual presentations:
The step S1 of providing a list 5 of studies including items suitable for identifying different studies.
The step S2 of providing a list 8, 21, 21′ of presentations including items suitable for identifying different kinds of presentations.
Items that indicate by their labeling the contrast that is inherent to a kind of presentations due the manner the presentations of the kind of presentations are generated are good examples of such items. Hence, the list of presentations may be a list of contrasts, for example.
The list 8, 21, 21′ of presentations is usually an organized list. For example, it is organized according to a workflow used by a practitioner during a medical evaluation of the body portion.
A step S10 of attributing first identifiers 40 to first virtual presentations 18 of the study 11 of the first kind and second identifiers 45 to second virtual presentations 18′ of the study 12 of the second kind.
Attributing first identifiers 40 to first virtual presentations 18 means that a unique identifier is attributed to each of the first virtual presentations 18 or, as it is usually the case, to a selection 29 of first virtual presentations of the first virtual presentations 18.
The identifier is not only unique with respect to the attributed first identifiers 40 but with respect to all identifiers attributed during the step S10 of attributing. In particular, the identifier is unique with respect to the first, second and, as the case may be, further identifiers attributed during the step S10 of attributing, wherein the further identifiers may be attributed to virtual presentations of a study of a further kind.
Usually, first identifiers 40 are attributed to a selection 29 of first virtual presentations of the first virtual presentations 18, only. Thereby, the selection is such that the selection 29 of first virtual presentations does not include any redundant information.
Further, the selection may be such that the selection 29 of first virtual presentations includes presentations of sufficient quality (e.g. no presentations that are blurred, for example due to patient movement during acquisition) and/or presentations of a given (e.g. pre-selected) set of contrasts, only.
Attributing second identifiers 45 to second virtual presentations 18′ means that a unique identifier is attributed to each of the second virtual presentations 18′ or, as it is usually the case, to a selection 29′ of second virtual presentations of the second virtual presentations 18′. The identifier is not only unique with respect to the attributed second identifiers 45 but with respect to all identifiers attributed during the step S10 of attributing. In particular, the identifier is unique with respect to the first, second and, as the case may be, further identifiers attributed during the step S10 of attributing, wherein the further identifiers may be attributed to virtual presentations of a study of a further kind.
Usually, second identifiers 45 are attributed to a selection 29′ of second virtual presentations of the second virtual presentations 18′, only. Thereby, the selection is such that the selection 29′ of second virtual presentations does not include any redundant information. Further, the selection may be such that the selection 29′ of second virtual presentations includes presentations of sufficient quality (e.g. no presentations that are blurred, for example due to patient movement during acquisition) and/or presentations of a given (e.g. pre-selected) set of contrasts, only.
It goes without saying that the step S10 of attributing may include attributing further identifiers (“third identifiers”, “fourth identifiers” . . . ) to virtual presentations (“third virtual presentations”, “fourth virtual presentations” . . . ) of studies of further kinds (“third kind”, “fourth kind” . . . ). In other words, the step S10 of attributing may further includes attributing nth identifiers to nth virtual presentations of a study of the nth kind, wherein this further attribution is carried out as disclosed with respect to the first identifiers 40 and second identifiers 45.
In the embodiment shown in FIG. 2, and in many embodiments of the method for navigating according to the first, second or third aspect of the invention, the step S10 of attributing includes the step S3 of assigning studies, this means the study 11 of the first kind and the study 12 of the second kind (and, as the case may be, studies of further kinds) to items of the list 5 of studies, wherein maximal one study is assigned to an item.
For example, every study that refers to the body portion of the patient, said body portion being the body portion to be evaluated, may be assigned each to exactly one item of the list 5 of studies but never to an item of the list 5 of studies to which another study is assigned.
It goes without saying that the study that is assigned to an item of the list 5 of studies is the study that corresponds to, for example has, the feature (characteristic) indicated by item to make the item suitable for identifying different studies.
In other words, the studies 11, 12 are assigned to their corresponding items of the list 5 of studies, this means to the items of the list 5 of studies suitable for identifying these studies 11, 12.
In the embodiment shown FIG. 2, and in many embodiments of the method for navigating according to the first, second or third aspect of the invention, the step S10 of attributing includes further the step S4 of assigning presentations, this means the first virtual presentations 18 or the selection 28 thereof and the second virtual presentations 18′ or the selection 28′ thereof (and, as the case may be, further virtual presentations or selections thereof), to items of the list 8, 21, 21′ of presentations, wherein maximal one presentation of the first virtual presentations 18 or the selection 28 thereof and maximal on presentation of the second virtual presentations 18′ or the selection 28′ (and, as the case may be, maximal one presentation of each study of a further kind or a selection thereof) are assigned to an item of the list 8, 21, 21′ of presentations.
It goes without saying that the presentations that are assigned to an item of the list 8, 21, 21′ of presentation are presentations of the kind to which the item refers. In other words, the presentations that are assigned to an item of the list 8, 21, 21′ of presentations have the feature (characteristic) that corresponds to the feature (characteristic) indicated by the item to make the item suitable for identifying different kinds of presentations.
For example, for each study assigned to an item of the list 5 of studies all presentations or all presentations of the selections thereof may be assigned to exactly one item of the list 8, 21, 21′ of presentations but never to an item of the list 8, 21, 21′ of presentations to which another presentation of the same study is assigned.
In other words, the first virtual presentations 18 or the selection 28 thereof and the second virtual presentations 18′ or the selection 28′ thereof are assigned to their corresponding items of the list 8, 21, 21′ of studies, this means to the items of the list 8, 21, 21′ of presentations suitable for identifying these kinds of presentations.
Further details of the step S10 of attributing unique identifiers, this means of attributing first identifiers 40 and second identifiers 45 in the embodiment of FIG. 2, are shown with respect to FIG. 3. In particular, details of the unique identifiers and of the link of each unique identifier to exactly one item of the list 5 of studies and to exactly one item of the list 8, 21, 21′ or presentations are shown.
The step S5 of providing, via a user interface 1, the list 5 of studies or a list of available studies and the list 8, 21, 21′ of presentations or a list of available kinds of presentations to a user, wherein the user interface allows for the selection of one item in the provided list 5 of studies and of one item in the provided list 8, 21, 21′ of presentations, wherein the selections can be changed.
The list of available kinds of presentations or at least its layout may be study-dependent.
Further, the user interface 1 is configured to display the presentation belonging to the study selected by selecting an item in the provided list 5 of studies and being of the kind selected by selecting an item in the provided list 8, 21, 21′ of presentations.
A step S11 of navigating through the first virtual presentations 29 having attributed a first identifier 40 and/or the second virtual presentations 29′ having attributed a second identifier 45.
The first virtual presentations 29 having attributed a first identifier 40 are all first virtual presentations to each of which a unique first identifier 40 was attributed in the step S10 of attributing.
The second virtual presentations 29′ having attributed a second identifier 45 are all second virtual presentations to each of which a unique first identifier 40 was attributed in the step S10 of attributing.
The step S11 of navigating includes the step S6 of selecting a presentation 3 to be displayed to the user by selecting an item in the list 5 of studies provided to the user and by selecting an item in the list 8, 21, 21′ of presentations provided to the user.
In particular, the user selects in a consecutive manner the presentations she/he wants to see by selecting items in the provided list 5 of studies and/or in the provided list 8, 21, 21′ of presentations. In other words, the user navigates in a very simple and intuitive manner through the presentations available for the body portion of the patient to be evaluated.

FIG. 3 shows a visualization of an exemplary embodiment of a background process that runs during execution of a method according to FIG. 1 or 2, for example. The background process includes various background steps that are described in detail further below.

The inputs to the background process are the dataset 50 provided in the step S8 of accessing (receiving, retrieving) a dataset, the list 5 of studies provided in the step S1 of providing a list of studies, and the list 8, 21, 21′ of presentations provided in the step S2 of providing a list of presentations.

The dataset 50 and the step S8 of accessing (receiving, retrieving) a dataset are as disclosed with respect to FIG. 1 or 2, for example.

In particular, the dataset 50 includes the study 11 of a first kind including the first virtual presentations 18 and the study 12 of a second kind including the second virtual presentations 18′.

The list 5 of studies and the step S1 of providing a list of studies are as disclosed with respect to FIG. 1 or 2, for example.

The list 8, 21, 21′ of presentations and the step S2 of providing a list of presentations are as disclosed with respect to FIG. 1 or 2, for example.

Two lists of presentations are shown in FIG. 3. This indicates that there may be a first list 21 of presentations related to the study 11 of the first kind and a second list 21′ of presentations related to study of the second kind (and, as the case may be, further lists of presentations related to studies of further kinds). However, more than one list of presentations is not a requirement. In many embodiments, there is only one list 8 of presentations, in particular only one list of presentations that lists the contrasts to be considered to assess a medical condition possibly shown in the studies 11, 12.

The embodiment shown in FIG. 3 includes further a step S9 of selecting virtual presentations from the virtual presentations of the studies to be considered. In the embodiment shown in FIG. 3, the step S9 of selecting is a step of selecting first virtual presentations from the first virtual presentations 18 to generate a selection 28 of first virtual presentations and of selecting second virtual presentations from the second virtual presentations 18′ to generate a selection 28′ of second virtual presentations.

The selections 28, 28′ of first/second virtual presentations includes only presentations that are relevant and useful for the medical assessment at hand. In particular, the selections do not include any redundant information or blurred presentations. In particular the selection 28 of first virtual presentations does not include first virtual presentations of the same kind of presentation (in particular of the same contrast) and the selection 28′ of second virtual presentations does not include second virtual presentations of the same kind of presentation.

The step S9 of selecting is usually an automated step having the dataset 50 as input and the selections 28, 28′ as output.

The embodiment of FIG. 3 includes further the step S3 of assigning studies, this means the study 11 of the first kind and the study 12 of the second kind (and, as the case may be, studies of further kinds) to items of the list 5 of studies. This step S5 is as disclosed with respect to FIG. 1 or 2, for example.

The step S3 of assigning studies is usually an automated step. For example, the assignment includes reading out the name of the studies 11, 12 and/or their metadata. In the exemplary embodiment shown in FIG. 3, the name of the study 11 of the first kind is P1_D1 (Patient 1, Date 1) and the name of the study 12 of the second kind is P1_D2 (Patient 1, Date 2). In this simplified example, the Date 1 and Date 2 would be read out.

The embodiment of FIG. 3 includes further the step S4 of assigning presentations, this means, in the embodiment shown in FIG. 3 the selection 28 of first virtual presentations and the selection 28′ of second virtual presentations (and, as the case may be, selections of further virtual presentations), to items of the list 8, 21, 21′ of presentations. This step S4 is as disclosed with respect to FIG. 1 or 2, for example.

The step S4 of assigning presentations is usually an automated step. For example, the assignment includes reading out the name of the selected presentations and/or their metadata. In the exemplary embodiment shown in FIG. 3, the names of the first virtual presentations comprised in the selection 28 of first virtual presentations are C1.3 (Contrast (kind) 1, acquirement 3), C2.1 (Contrast (kind) 2, acquirement 1), C3.2 (Contrast (kind) 3, acquirement 2) and the names of the second virtual presentations comprised in the selection 28′ of second virtual presentations are C1.1 (Contrast (kind) 1, acquirement 1), C2.3 (Contrast (kind) 2, acquirement 3), C4.1 (Contrast (kind) 4, acquirement 1). In this simplified example, the contrasts (kinds) 1, 2, 3 would be read out in the case of the study 11 of the first kind and the contrasts (kinds 1, 2, 4 would be read out in the case of the study 12 of the second kind.

The embodiment of FIG. 3 includes further a step S10 of attributing first identifiers 40 to first virtual presentations 18 of the study 11 of the first kind, more precisely to the first virtual presentations of the selection 28 of first virtual presentations, and second identifiers 45 to second virtual presentations 18′ of the study 12 of the second kind, more precisely to the second virtual presentations of the selection 28′ of second virtual presentations. This step S10 of attributing corresponds to the step S10 of attributing disclosed with respect to FIG. 2, with the exception that the step S3 of assigning studies and the step S4 of assigning are presented as independent steps in FIG. 3 and as sub-steps of the step S10 of attributing in FIG. 2.

In the embodiment shown in detail in FIG. 3, an index is attributed to each item of the list 5 of studies, this index being unique within the indexes (1, 2, 3, 4 . . . in FIG. 3) attributed to items of the list 5 of studies.

Further, an index is attributed to each item of the list(s) 8, 21, 21′ of presentations, this index being unique within the indexes (a, b, c, d . . . in FIG. 3) attributed to items of the list(s) 8, 21, 21′ of presentations.

The first identifiers 40 are created then by attributing, to each first virtual presentation of the selection 28 of first virtual presentation, the index of the item of the list 5 of studies to which the study 11 of the first kind is attributed (first sub-identifier 41) and the index of the item of the list 8, 21 of presentations to which this first virtual presentation is attributed (second sub-identifier 42).

Accordingly, the second identifiers 45 are created by attributing, to each second virtual presentation of the selection 28′ of second virtual presentation, the index of the item of the list 5 of studies to which the study 12 of the second kind is attributed (third sub-identifier 46) and the index of the item of the list 8, 21′ of presentations to which this second virtual presentation is attributed (second sub-identifier 47).

The first virtual presentations 29 having attributed a first identifier 40 and the second virtual presentations 29 having attributed a second identifier 45 are linked one-to-one to the items of the list 5 of studies and the items of the list 8, 29, 29′ or presentations via the attributed identifiers that are unique identifiers. This, in combination with the selection made in the step S9 of selecting, allows for the fast and efficient navigation through the relevant virtual presentations of the study 11 of the first kind and the study 12 of the second kind.

The step S10 of attributing, including the creation of the unique identifiers, is an automated step.

In the embodiment shown in FIG. 3, the first virtual presentations 29 having attributed a first identifier 40 are the first virtual presentations of the selection 28 of first virtual presentations and the second virtual presentations 29′ having attributed a second identifier 45 are the second virtual presentations of the selection 28′ of second virtual presentations. This, however, is no requirement. Rather, the first virtual presentations 29 having attributed a first identifier 40 may be a part of the first virtual presentations of the selection 28 of first virtual presentations. In particular, this may be the case if a first virtual presentation of the selection 28 of first virtual presentations is of a kind (contrast) to which no item of the list 8, 21 of presentations corresponds, for example because this kind of presentation is considered as not relevant or helpful. Accordingly, the second virtual presentations 29′ having attributed a second identifier 45 may be a part of the second virtual presentations of the selection 28′ of second virtual presentations.

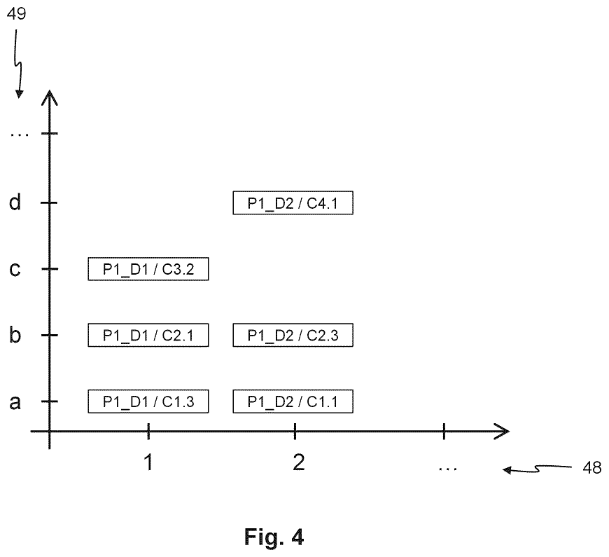

FIG. 4 shows a visualization of the data structure resulting from the background processes according to FIG. 3.

The resulting data structure is a grid-like data structure that is spanned by the sub-identifiers attributed to an index of an item of the list 5 of studies (sub-identifiers 48 of the first kind) and by the sub-identifiers attributed to an index of an item of the list 8, 21, 21′ of presentations (sub-identifiers 49 of the second kind).

It goes without saying that the data structure resulting from the use of the created unique identifiers 40, 45 is non-hierarchical. In particular, the kind (contrast) of a presentation is not on a lower hierarchical level than the kind (e.g. date) of a study and vice versa.

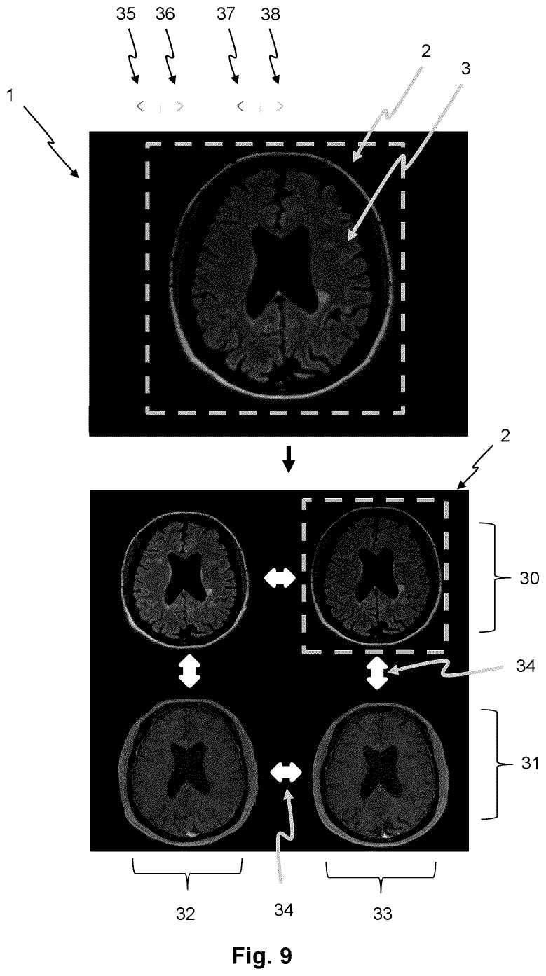

Further, it is evident from a comparison of FIG. 4 with FIG. 9 (in particular lower part of FIG. 9) that this data structure allows directly for the one-click or one-key operations shown in FIG. 9.

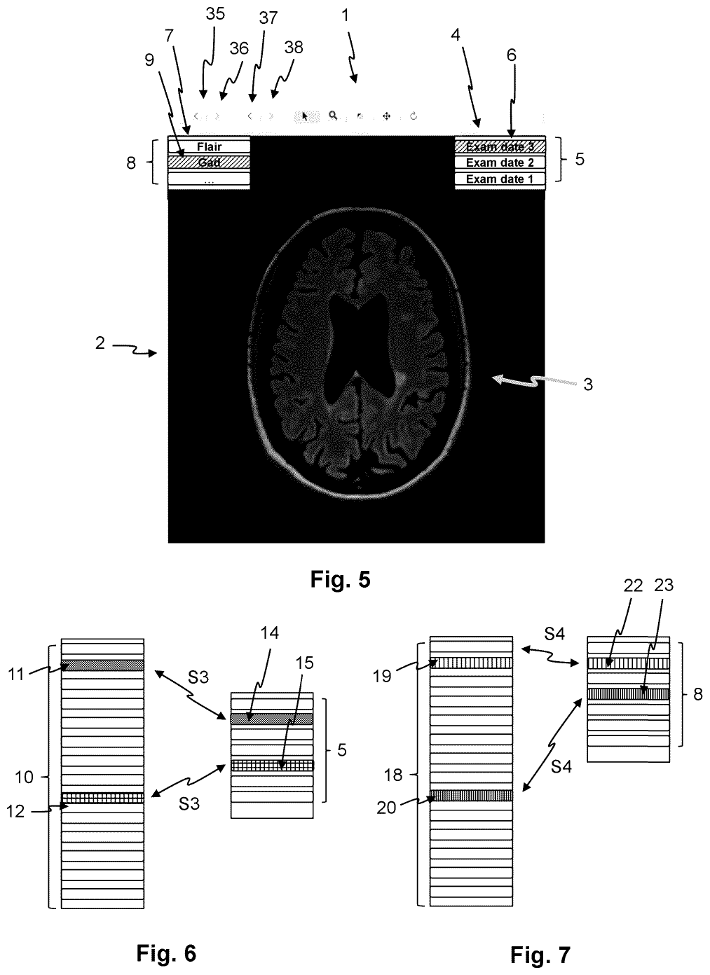

FIG. 5 shows an exemplary embodiment of a user interface 1 that is configured for use in any disclosed method for navigating through virtual presentations and/or for selecting and displaying virtual presentations, for example in one of the methods disclosed with respect to FIG. 1 or 2.

The user interface 1 includes an image viewer 2, in which the presentation selected by selecting an item in the provided list 5 of studies and an item in the provided list 8 of presentations is displayed (displayed presentation 3).

The user interface 2 includes further a first view 4 in which the list 5 of studies is provided and a second view 7 in which the list 8 of presentations is provided.

First and second views are usually active views in the sense that an item of the lists shown in the views can be selected by clicking on it or by touching it, for example.

The first and second views are usually further configured to highlight the currently selected study 6 and the currently selected kind of presentation 9.

The user interface 1 has usually further tools that allow easy and intuitive navigation through the presentations available for the body portion of the patient to be evaluated, for example toolbar buttons, such as a toolbar button 35 to display the presentation assigned to one back (with respect to the currently selected kind of presentation 9) in the list 7 of presentations (and belonging to currently selected study 6), a toolbar button 36 to display the presentation assigned to one forwards (with respect to the currently selected kind of presentation 9) in the list 7 of presentations (and belonging to currently selected study 6), a toolbar button 37 to display the presentation corresponding to one back (with respect to the currently selected study 6) in the list 5 of studies (and being of the currently selected kind of presentation 9), and a toolbar button 38 to display the presentation corresponding to one forwards (with respect to the currently selected study 6) in the list 5 of studies (and being of the currently selected kind of presentation 9).

FIG. 6 shows a visualization of an exemplary embodiment of the step S3 of assigning studies to the list 5 of studies.

According to the embodiment of FIG. 6, all studies 10 of a patient, or all studies 10 of the patient concerning a specific body portion, are accessed and the studies that correspond to an item of the list 5 of studies are assigned to the corresponding item as disclosed with respect to FIGS. 1-3.

In a variant of the method that may also apply in the methods according to FIGS. 1-3, the list 5 of studies is provided in an automated manner by accessing all studies of a patient and by identifying the studies of interest for the medical condition of the patient to be assessed. For example, all studies of a specific body portion of the patient and/or all studies having been generated in a given period of time are identified and items of the list 5 of studies are generated, said items being suitable for identifying the study that has initiated the generation of the item. Again, the labelling of the item may include the date of the study.

FIG. 6 shows schematically how, in a step S3 of assigning studies to the list 5 of studies, a first study 11 of all studies 10 is assigned to the corresponding item 14 of the list 5 of studies and how a second study 12 of all studies 10 is assigned to the corresponding item 15 of the list 5 of studies.

FIG. 7 shows a visualization of an exemplary embodiment of the step S4 of assigning presentations to the list 8 of presentations.

According to the embodiment of FIG. 7, all presentations 18 of a study that is assigned to an item of the list 5 of studies are accessed and the presentations that correspond to an item of the list 8 of presentations are assigned to the corresponding item as disclosed with respect to FIG. 1-3.

In a variant of the method that may also apply in the methods according to FIGS. 1-3, the list 8 of presentations is provided in an automated manner by accessing all studies assigned to an item of the list 5 of studies and by identifying the kind of presentations available in the studies. An item of the list 8 of presentations is then generated for each identified kind of presentation. Again, the labelling of the item may include a hint towards the contrast of the kind of presentation related to the item.

FIG. 7 shows schematically how, in a step S4 of assigning presentations to the list 8 of presentations, a first presentation 19 of all presentations 18 is assigned to the corresponding item 22 of the list 8 of presentations and how a second presentation 20 of all presentations 18 is assigned to the corresponding item 23 of the list 8 of presentations.

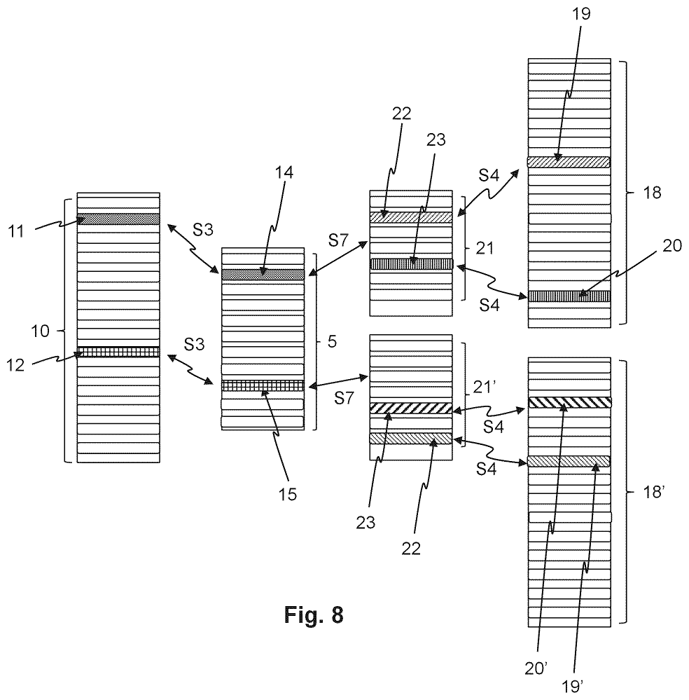

FIG. 8 shows a visualization of an exemplary embodiment of the steps of assigning studies to the list 5 of studies and of assigning presentations to lists 21, 21′ of presentations.

The embodiment the step S3 of assigning studies and of the step S4 of assigning presentations according to FIG. 8 distinguishes from the embodiments of these steps shown in FIGS. 6 and 7 in that a list 21, 21′ of presentations is provided for each study 11, 12 assigned to an item of the list 5 of studies. In other words, the method according to FIG. 8 includes a step S7 of providing a list 21, 21′ of presentations for each study assigned to an item of the list 5 of studies.

The lists 21, 21′ of presentations provided in said step S7 may be according to any list 8, 21, 21′ of presentations disclosed with respect to FIGS. 1-3, 5-7.

The step S7 of providing a list 21, 21′ of presentations for each study assigned to an item of the list 5 of studies may be an automated step. For example, the lists 21, 21 of presentations may be generated by accessing the assigned studies and by identifying the kind of presentations available in the studies.

FIG. 9 shows a visualization of an exemplary embodiment of navigating in the presentations of a plurality of studies and of a plurality of kinds of presentations.

The upper part of FIG. 9 shows a selection of elements of the user interface 1 shown in FIG. 5. In particular, the image viewer 2 in which the displayed presentation 3 is arranged and the toolbar buttons 35-38 for navigating back and forth in the list 5 of studies provided in the first view 4 and the list 8 of presentations provided in the second view 7 are shown.

The lower part of FIG. 9 visualizes how a presentation of a specific study and being of a specific kind of presentation is displayed in the image viewer 2 by one-click or one-key operations 34.

Each presentation assigned to an item of the list 8 of presentation is also assigned, via the study to which it belongs, to an item of the list 5 of studies. This makes a clear identification, and hence display in the image viewer 2, of each presentation assigned to an item of the list 8 of presentation possible.

For visualization, FIG. 9 shows two presentations 30 assigned to a first item in the list 8 of presentations and two presentations 31 assigned to a second item in the list 8 of presentations. One of the presentations 30 assigned to the first item and one presentation assigned to the second item are presentations 32 assigned further to a first item in the list 5 of studies. The other presentation of the presentations 30 assigned to the first item of the list 8 of presentations and the other presentation of the presentations 31 assigned to the second item of the list 8 of presentations are presentations 33 assigned further to a second item in the list 5 of studies.

### REFERENCE SIGNS

1 user interface
2 image viewer
3 displayed presentation
4 first view (view for the list of studies)
5 list of studies
6 selected study
7 second view (view for the list of series)
8 list of presentations
9 selected kind of presentation
10 all studies of one patient
11 one study, study of a first kind
12 another study, study of a second kind
14 an item of the list of studies to which a study is assigned
15 another item of the list of studies to which another study is assigned
18 all presentations of one study, first virtual presentations (all presentations of the study of a first kind)
18′ all presentations of another study, second virtual presentations (all presentations of the study of a second kind)
19 one presentation, one presentation of the study of a first kind
19′ one presentation, one presentation of the study of a second kind
20 another presentation, another presentation of the study of a first kind
20′ another presentation, another presentation of the study of a second kind
21 list of presentations of a corresponding study, list of presentations of the study of a first kind
21′ list of presentations of a corresponding study, list of presentations of the study of a second kind
22 an item of the list of presentations to which a presentation is assigned
23 another item of the list of presentations to which another presentation is assigned
28 selection of the first virtual presentations
28′ selection of the second virtual presentations
29 first virtual presentations having attributed a first identifier
29′ second virtual presentations having attributed a second identifier
30 presentations corresponding to one item of the list of presentations
31 presentations corresponding to another item of the list of presentations
32 presentations corresponding to one item of the list of studies
33 presentations corresponding to another item of the list of studies
34 1-click/1-touch/1-key operation
35 toolbar button to display the presentation corresponding to one back in the list of presentations
36 toolbar button to display the presentations corresponding to one forward in the list of presentations
37 toolbar button to display the presentation corresponding to one back in the list of studies
38 toolbar button to display the presentations corresponding to one forward in the list of studies
40 first identifier
41 first sub-identifier
42 second sub-identifier
45 second identifier
46 third sub-identifier
47 fourth sub-identifier
48 sub-identifiers of the first kind
49 sub-identifiers of the second kind
50 dataset
S1 Providing a list of studies
S2 Providing a list of presentations
S3 Assigning a study to an item of the list of studies
S4 Assigning a presentation to an item of the list of presentations
S5 Providing the list of studies and the list of presentations to a user
S6 Selecting a presentation by selecting an item of the provided list of studies and an item of the provided list of presentations
S7 Providing a list of presentations for each study assigned to an item of the list of studies
S8 Providing/accessing a dataset
S9 Selecting virtual presentation from the virtual presentations of a study
S10 Attributing first and second identifiers
S11 Navigating

1. A computer-implemented method for navigating through virtual presentations of a body portion, wherein the virtual presentations being generated in medical studies, the method comprises:
a step of providing a list of studies comprising at least one item that is characteristic for a kind of study;
a step of providing a list of presentations comprising at least one item that is characteristic for a kind of presentation;
a step of providing a dataset, wherein the dataset comprises a study of a first kind of study comprising first virtual presentations and wherein the dataset comprises a study of a second kind of study comprising second virtual presentations;
a step of attributing first identifiers to the first virtual presentations or to a selection thereof and second identifiers to the second virtual presentations or to a selection thereof,
wherein a first identifier comprises a first sub-identifier of sub-identifiers of a first kind that represent the items of the list of studies in a unique manner and a second sub-identifier of sub-identifier of a second kind that represent the items of the list of presentations in a unique manner, wherein the first sub-identifier is equal for all first identifiers,
wherein a second identifier comprises a third sub-identifier being a sub-identifier of the sub-identifier of the first kind and a fourth sub-identifier being a sub-identifier of the sub-identifier of the second kind,
wherein the third sub-identifier is equal for all second identifiers,
wherein the first sub-identifier is different from the third sub-identifier;
a step of providing to a user via a user interface the list of studies or an adapted list of studies and the list of presentations or an adapted list of presentations,
wherein the user interface comprises an image viewer, a first view for the list of studies or the adapted list of studies, and a second view for the list of presentations or the adapted list of presentations;
a step of navigating through at least one of the first virtual presentations having attributed a first identifier and the second virtual presentations having attributed a second identifier by selecting an item of the list shown in the first view and by selecting an item of the list shown in the second view,
wherein the step of navigating comprises displaying a virtual presentation,
wherein the displayed virtual presentation has the sub-identifier of the sub-identifier of the first kind that represents the item selected in the list of studies provided in the first view and the sub-identifier of the sub-identifier of the second kind that represents the item selected in the list of presentations provided in the second view.

2. A computer-executable method for creating unique identifiers for navigating through virtual presentations of a body portion, the virtual presentations being generated in medical studies, the method comprises:
a step of providing a list of studies comprising at least one item that is characteristic for a kind of study;
a step of providing a list of presentations comprising at least one item that is characteristic for a kind of presentation;
a step of generating first identifiers, wherein a first identifier comprises a first sub-identifier of sub-identifier of a first kind that represent the items of the list of studies in a unique manner and a second sub-identifier of sub-identifier of a second kind that represent the items of the list of presentations in a unique manner.

3. The method according to claim 2, wherein the first sub-identifier is equal for all first identifiers, wherein the method comprises a step of generating second identifiers, wherein a second identifier comprises a third sub-identifier that is a sub-identifier of the sub-identifier of the first kind and a fourth sub-identifier that is a sub-identifier of the sub-identifier of the second kind, wherein the third sub-identifier is equal for all second identifiers, and wherein the first sub-identifier is different from the third sub-identifier.

4. The method according to claim 3 comprising:
a step of accessing a dataset, wherein the dataset comprises a study of a first kind comprising first virtual presentations and wherein the dataset comprises a study of a second kind comprising second virtual presentations.

5. The method according to claim 4 comprising:
a step of attributing first identifiers to the first virtual presentations or to a selection thereof and second identifiers to the second virtual presentations or to a selection thereof,
wherein the step of attributing comprises a step of assigning the study of the first kind to its corresponding item of the list of studies and of assigning the study of the second kind to its corresponding item of the list of studies, and a step of assigning the first virtual presentations or the selection thereof to their corresponding items of the at least one item of the list of presentations and of assigning the second virtual presentations or the selection thereof to their corresponding items of the at least one item of the list of presentations.

6. A computer-executable method for navigating through virtual presentations of a body portion, the virtual presentations being generated in medical studies, the method comprises:
a step of creating unique identifiers using the method according to claim 4;
a step of providing to a user via a user interface the list of studies or an adapted list of studies and the list of presentations or an adapted list of presentations,
wherein the user interface comprises an image viewer, a first view for the list of studies or the adapted list of studies, and a second view for the list of presentations or the adapted list of presentations;
a step of navigating through at least one of the first virtual presentations having attributed a first identifier and the second virtual presentations having attributed a second identifier by selecting an item of the list shown in the first view and by selecting an item of the list shown in the second view;
wherein the step of navigating comprises displaying a virtual presentation, wherein the displayed virtual presentation has the sub-identifier of the sub-identifiers of the first kind that represents the item selected in the list of studies provided in the first view and the sub-identifier of the sub-identifiers of the second kind that represents the item selected in the list of presentations provided in the second view.

7. The method according to claim 1, wherein the list shown in the first view is an ordered list, and the list shown in the second view is an ordered list.

8. The method according to claim 1, wherein the presentations having attributed a first or second identifier are linked to the items of the list shown in the first view and the items of the list shown in the second view via the attributed identifier.

9. The method according to claim 1, comprising a step of providing a list of presentations for each study to be navigated in, wherein the list shown in the second view is the list of presentations or the adapted list of presentations that corresponds to the study selected by selecting an item of the list shown in the first view.

10. The method according to claim 1, wherein the selection of another item of the list shown in the first view than the currently selected item occurs at a fixed item in the list shown in the second view, and the selection of another item of the list shown in the second view than the currently selected item occurs at a fixed item in the list shown in the first view.

11. The method according to claim 1, wherein the user interface comprises tools allowing the user to at least one of:
replace the presentation displayed in the image viewer with a replacement presentation that corresponds to the presentation assigned to the item arranged in the list shown in the second view precedingly or subsequently to the item to which the presentation displayed in the image viewer is assigned;
replace the presentation displayed in the image viewer with a replacement presentation that belongs to the study assigned to the item arranged in the list shown in the first view precedingly or subsequently to the item to which the study is assigned to which the presentation displayed in the image viewer belongs;
switch back and forth between displaying in the image viewer a first presentation and a second presentation, wherein the first presentation is a presentation assigned to a first item in the list shown in the second view and the second presentation is a presentation assigned to a second item in the list shown in the second view;
switch back and forth between displaying in the image viewer a first presentation and a second presentation, wherein the first presentation is a presentation of the study assigned to a first item in the list shown in the first view and the second presentation is a presentation of the study assigned to a second item in the list shown in the first view.

12. The method according to claim 1, wherein the items of the list of presentations are suitable for identifying presentations of different kinds of presentation by the items referring to settings available or used during acquisition of raw data or presentations and/or by the items referring to features available or used during processing of acquired raw data or acquired presentations.

13. The method according to claim 1, wherein the list of studies comprises unique items only and the list of presentations comprises unique items only.

14. The method according to claim 1, wherein the virtual presentations having attributed a first or second identifier are coregistered to one another, to one of the virtual presentations, or to a reference presentation.

15. The method according to claim 14, wherein the virtual presentations having attributed a first or second identifier are coregistered to a reference presentation and wherein an algorithm determines the reference presentation from a set of virtual presentations that comprises all presentations having attributed a first or second identifier.

16. An evaluation system for virtual presentations of a body portion, the virtual presentations being generated in medical studies, the system comprises a memory, a communication unit, a controller, and a user interface, wherein;
a list of studies comprising at least one item that is characteristic for a kind of study and a list of presentations comprising at least one item that is characteristic for a kind of presentation, the list of studies and the list of presentations being stored in the memory;
the communication unit is configured to access a dataset, wherein the dataset comprises a study a first kind comprising first virtual presentations and wherein the dataset comprises a study of a second kind comprising second virtual presentations,
wherein first identifiers are attributed to the first virtual presentations or to a selection thereof,
and wherein second identifiers are attributed to the second virtual presentations or to a selection thereof,
wherein a first identifier comprises a first sub-identifier of sub-identifiers of a first kind that represent the items of the list of studies in a unique manner and a second sub-identifier of sub-identifiers of a second kind that represent the items of the list of presentations in a unique manner, wherein the first sub-identifier is equal for all first identifiers,
wherein a second identifier comprises a third sub-identifier being a sub-identifier of the sub-identifiers of the first kind and a fourth sub-identifier being a sub-identifier of the sub-identifiers of the second kind, wherein the third sub-identifier is equal for all second identifiers,
wherein the first sub-identifier is different from the third sub-identifier;
the user interface comprises an image viewer, a first view for providing the list of studies or an adapted list of studies, and a second view for providing the list of presentations or an adapted list of presentations,
wherein the user interface is configured to receive a user input, wherein the user input comprises selecting an item of the list shown in the first view and an item of the list shown in the second view;
the controller is configured to control the evaluation system such that a virtual presentation is displayed in the image viewer, wherein the displayed virtual presentation is the virtual presentation that has the sub-identifier of the sub-identifiers of the first kind that represents the item selected in the list of studies provided in the first view and the sub-identifier of the sub-identifiers of the second kind that represents the item selected in the list of presentations provided in the second view.

17. A system for navigating through virtual presentations of a body portion, wherein the virtual presentations being generated in medical studies, the system comprises a controller and a user interface, wherein the user interface comprises an image viewer, a first view for providing a list of studies or an adapted list of studies, a second view for providing a list of presentations or an adapted list of presentations, and tools for selecting an item of the list shown in the first view and an item of the list shown in the second view, and wherein the system is configured to carry out a method according to claim 1.

18. A computer program comprising instructions which, when the program is executed by a computer, cause the computer to execute the steps of the method according to claim 1.

19. A reproducible computer-readable signal encoding the computer program according to claim 18.
